(19) United States
(12) Patent Application Publication
(10) Pub. No.: US 2007/0021918 A1
(43) Pub. Date: Jan. 25, 2007

(54) Universal gene chip for high throughput chemogenomic analysis

(76) Inventors: Georges Natsoulis, Kensington, CA (US); Leslie J. Browne, Pennington, NJ (US)

Correspondence Address: HOWREY LLP, C/O IP DOCKETING DEPARTMENT, 2941 FAIRVIEW PARK DRIVE, SUITE 200, FALLS CHURCH, VA, 22042-2924, US

(21) Appl. No.: 11/114,998

(22) Filed: Apr. 25, 2005

Related U.S. Application Data
(60) Provisional application No. 60/565,793, filed on Apr. 26, 2004.

(51) Int. Cl. G01N 33/48; G01N 33/50; G06F 19/00

(52) U.S. Cl. 702/19

(57) ABSTRACT

The invention provides methods for preparing reagent sets based on small subsets of highly informative genes capable of carrying out a broad range of chemogenomic classification tasks. The invention also provides high-throughput diagnostic assays and devices based on these reduced subsets of information rich genes. In addition, the invention provides a general method for selecting a reduced subset of highly responsive variables from a much larger multivariate dataset, and thus, use of these variables to prepare diagnostic measurement devices, or other analytic tools, with little or no loss of performance relative to devices or tools incorporating the full set of variables.

### CROSS REFERENCE TO RELATED APPLICATION

This application claims priority from U.S. Provisional Application No. 60/565,793, filed Apr. 26, 2004, which is hereby incorporated by reference in its entirety.

### TABLES SUBMITTED ON CD

This application includes a CD containing the ASCII format files named “Table_{—}2.txt” “Table_{—}4.txt” and “Table_{—}7.txt” that are 105 kB, 56 kB, and 386 kB, in size, respectively. These files (and the CD) were created on Apr. 25, 2005. This CD, and the files thereon, which contain Tables 2, 4 and 7 referred to in the text below, is hereby incorporated by reference herein.

### FIELD OF THE INVENTION

The invention relates to methods for providing small subsets of highly informative genes sufficient to carry out a broad range of chemogenomic classification tasks. The invention also provides high-throughput assays and devices based on these reduced subsets of information rich genes. In addition, the invention provides a general method for selecting a reduced subset of highly responsive variables from a much larger multivariate dataset, and thus use of these variables to prepare diagnostic measurement devices, or other analytic tools, with little or no loss of performance relative to devices or tools incorporating the full set of variables.

### BACKGROUND OF THE INVENTION

A diagnostic assay typically consists of performing one or more measurements and then assigning a sample to one or more categories based on the results of the measurement(s). Desirable attributes of a diagnostic assay include high sensitivity and specificity measured in terms of low false negative and false positive rates and overall accuracy. Because diagnostic assays are often used to assign large number of samples to given categories, the issues of cost per assay and throughput (number of assays per unit time or per worker hour) are of paramount importance.

Usually the development of a diagnostic assay involves the following steps: (1) define the end point to diagnose, (e.g., cholestasis, a pathology of the liver); (2) identify one or more measurements whose value correlates with the end point, (e.g., elevation of bilirubin in the bloodstream as an indication of cholestasis); and (3) develop a specific, accurate, high-throughput and cost-effective device for making the specific measurements needed to predict or determine the endpoint. In order to increase throughput and decrease costs several diagnostic assays are often combined in a single device (e.g., an assay panel), especially when the detection methodologies are compatible. For example several ELISA-based assays, each using a different antibody to ascertain a different end point may be combined in a single panel and commercialized as a single kit. Even in this case, however, each of the different antibody based assays first had to be developed individually, and required the generation of one or more specific reagents.

Over the past 10 years, a variety of techniques have been developed that are capable of measuring a large number of different biological analytes simultaneously but which require relatively little optimization for any of the individual analyte detectors. Perhaps the most successful example is the DNA microarray, which may be used to measure the expression levels of thousands or even tens of thousands of genes simultaneously. Based on well-established hybridization rules, the design of the individual probe sequences on a DNA microarray now may be carried out in silico, and without any specific biological question in mind. Although DNA microarrays have been used primarily for pure research applications, this technology currently is being developed as a medical diagnostic device and everyday bioanalytical tool.

A more recently developed powerful new application for the DNA microarray is chemogenomic analysis. The term “chemogenomics” refers to the transcriptional and/or bioassay response of one or more genes upon exposure to a particular chemical compound. A comprehensive database of chemogenomic annotations for large numbers of genes in response to large numbers of chemical compounds may be used to design and optimize new pharmaceutical lead compounds based only on a transcriptional and biomolecular profile of the known (or merely hypothetical) compound. For example, a small number of rats may be treated with a novel lead compound and then expression profiles measured for different tissues from the compound treated animals using DNA microarrays. Based on the correlative analysis of this compound treatment expression level data with respect to the chemogenomic reference database, it may be possible to predict the toxicological profile and/or likely off-target effects of the new compound. Construction of a comprehensive chemogenomic database and methods for chemogenomic analysis using microarrays are described in Published U.S. Pat. Appl. No. 2005/0060102 A1, which is hereby incorporated herein by reference in its entirety.

Although DNA microarrays are considerably more expensive than conventional diagnostic assays they do offer two critical advantages. First, they tend to be more sensitive, and therefore more discriminating and accurate in prediction than most current diagnostic techniques. Using a DNA microarray, it is possible to detect a change in a particular gene's expression level earlier, or in response to a milder treatment than is possible with more classical pathology markers. Also, it is possible to discern combinations of genes or proteins useful for resolving subtle differences in forms of an otherwise more generic pathology. Second, because of their massively parallel design, DNA microarrays make it possible to answer many different diagnostic questions using the data collected in a single experiment.

The challenge in using a DNA microarray as a diagnostic tool lies in the interpretation of the large amount of multivariate data provided by each measurement (i.e. each probe's hybridization). Indeed, commercially available high density DNA microarrays (also referred to as “gene chips” or “biochips”) allow one to collect thousands of gene expression measurements using standardized published protocols. However, typically only a very small fraction of these measurements are relevant to a given diagnostic question being asked by the user. Thus, current DNA microarrays provide a burdensome amount of information when answering most typical diagnostic assay questions. Similar data overload problems exist in adapting other highly multiplexed bioassays such as RT-PCR or proteomic mass spectrometry to diagnostic applications.

Generally, statistical techniques have been used to address the data overload problems associated with the use of massively multiplexed assays, like DNA microarrays, RT-PCR and proteomic mass spectrometric assays. For example, this problem has been addressed to some extent using supervised clustering methods and supervised two-class classification methods such as support vector machines (SVMs), decision trees, logistic regression, and neural nets (see, e.g., Hastie, T., R. Tibshirani, and J. Friedman. 2001. Elements of statistical learning: data mining, inference and prediction. Springer-Verlag). Statistical methods for dealing with torrents of data, however, cannot solve the fundamental problem of excessive time and cost associated with preparing (or buying) and processing these highly complex measurement devices. Commercially available high density DNA microarrays are expensive. A typical single use commercial DNA microarray with 10,000 genes costs on the order of $500 and the associated instrumentation and computers necessary to acquire, store and manipulate the data further add to the costs. High-throughput proteomic analysis systems are even more expensive when considered on a per data point basis. A single high quality mass spectrometer for high-throughput proteomic analysis costs in excess of $500,000.

The problem remains that sifting through the massive amounts of multivariate data produced by highly multiplexed devices(such as DNA microarrays) to identify those variables useful for answering a few specific diagnostic questions remains a difficult problem. Thus, there is a need for lower cost versions of DNA microarrays and other high-throughput devices useful for chemogenomic analysis and other types of diagnostic measurements. Of particular value would be methods for identifying a small subset of information rich variables (e.g., specific sets of genes or proteins) that are still capable of answering a full range of diagnostic questions.

### SUMMARY OF THE INVENTION

In one embodiment, the present invention provides a method for preparing a high-throughput chemogenomic assay reagent set comprising: (1) deriving a set of non-redundant classifiers, each comprising a plurality of genes, from a chemogenomic dataset, wherein the chemogenomic dataset comprises expression levels for a plurality of gene measured in response to a plurality of compound treatments; (2) ranking each gene in the set of non-redundant classifiers based on its contribution across all of the non-redundant classifiers; (2) selecting the subset of genes ranking in about the 50^{th} percentile or higher; and (4) preparing a subset of isolated polynucleotides or polypeptides, wherein each polynucleotide or polypeptide in the subset is capable of detecting a different one of the selected genes.

In other embodiments, the above described method for preparing a high-throughput chemogenomic assay reagent set may be carried out wherein the chemogenomic dataset comprises expression levels for at least about 1000, at least about 5000, or at least about 10,000 genes. In other embodiments, the method may be carried out wherein the chemogenomic dataset comprises at least about 50, at least about 100, or at least about 500 different compound treatments. In other embodiments, the method may be carried out wherein the selected subset of genes ranks in at least about the 60^{th}, 70^{th}, 80^{th}, 90^{th}, or 95^{th} percentile or higher. In other embodiments, the method may be carried out wherein the selected subset of genes comprises about 1000, about 800, about 500, about 200, or about 100 or fewer genes. In other embodiments, the method may be carried out wherein the selected subset of genes comprises as few as about 20%, about 10%, about 5%, about 2%, or even about 1% or fewer of the genes in the chemogenomic dataset.

In other embodiments, the above described method for preparing a high-throughput chemogenomic assay reagent set may be carried out wherein the method of ranking the genes across all classifiers is selected from the group consisting of: determining the sum of weights; determining the sum of absolute value of weights; and determining the sum of impact factors. In other embodiments, the method may be carried out wherein the set of non-redundant classifiers comprises at least about 50, at least about 100, or at least about 200 classifiers. In other embodiments, the method may be carried out wherein the redundancy of the classifiers is determined using a fingerprint of resulting classifiers against a set of reference treatments, and in some embodiments, the fingerprint is assessed using a hierarchical clustering method selected from the group consisting of: UPGMA, WPGMA, a correlation coefficient distance metric, and a Euclidian distance metric. In addition, the present invention provides reagent sets made according to a method comprising: (1) deriving a set of non-redundant classifiers, each comprising a plurality of genes, from a chemogenomic dataset, wherein the chemogenomic dataset comprises expression levels for a plurality of gene measured in response to a plurality of compound treatments; (2) ranking each gene in the set of non-redundant classifiers based on its contribution across all of the non-redundant classifiers; (2) selecting the subset of genes ranking in about the 50^{th} percentile or higher; and (4) preparing a subset of isolated polynucleotides or polypeptides, wherein each polynucleotide or polypeptide in the subset is capable of detecting a different one of the selected genes. In other embodiments, the invention provides reagent sets made according to the above method wherein the number of reagents in the subset is less than about 10% of the number of genes in the full chemogenomic dataset. In another embodiment, the number of reagents in the subset is less than about 5% of the number of genes in the full chemogenomic dataset. In another embodiment the number of genes in the subset is about 800, about 600, about 400, about 200, or about 100 or fewer.

The present invention also provides an array comprising a reagent set made according to the method comprising: (1) deriving a set of non-redundant classifiers, each comprising a plurality of genes, from a chemogenomic dataset, wherein the chemogenomic dataset comprises expression levels for a plurality of gene measured in response to a plurality of compound treatments; (2) ranking each gene in the set of non-redundant classifiers based on its contribution across all of the non-redundant classifiers; (2) selecting the subset of genes ranking in about the 50^{th} percentile or higher; and (4) preparing a subset of isolated polynucleotides or polypeptides, wherein each polynucleotide or polypeptide in the subset is capable of detecting a different one of the selected genes. In other embodiments, the invention provides reagent sets made according to the above method wherein the number of reagents in the subset is less than about 10% of the number of genes in the full chemogenomic dataset. In one array embodiment of the invention, the reagent set consists of polynucleotides capable of detecting the genes listed in Table 4. In another array embodiment, the reagent set consists of polynucleotides capable of detecting the top ranking 800 genes listed in Table 4. In another array embodiment, the reagent set consists of polypeptides each capable of detecting a secreted protein encoded by the genes listed in Table 5.

In another embodiment, the invention provides a reagent set for chemogenomic analysis of a compound treated sample, wherein the set comprises a plurality of polynucleotides or polypeptides, wherein each polynucleotide or polypeptide is capable of detecting at least one member of a subset of less than about 10 percent of the genes in a full chemogenomic dataset, and wherein the subset of genes is capable of generating a set of signatures that exhibit at least about 85 percent of the average performance of the same set of signatures generated from the full chemogenomic dataset. In one preferred embodiment, the reagent set comprises a plurality of polynucleotides. In one embodiment, the reagent is generated from a full chemogenomic dataset that comprises expression levels for at least about 5000, about 8000, or about 10,000 genes. In one embodiment, the reagent is generated from a full chemogenomic dataset comprises at least about 100, about 300, about 500, about 1000, or about 1500 different compound treatments. In one embodiment, the invention provides a reagent set wherein the subset comprises less than about 5%, about 3%, or about 1% of the genes in the full chemogenomic dataset. In one embodiment, the invention provides a reagent set wherein the set of signatures comprises at least about 25, about 50, about 75, about 100, or at least about 125 signatures. In one preferred embodiment, the invention provides a reagent set wherein the signatures are linear classifiers generated using support vector machines. In another embodiment, the invention provides reagent sets wherein the subset is capable of generating a set of signatures that exhibit at least about 95 percent of the average performance of the same set of signatures generated from the full chemogenomic dataset. In another embodiment, the invention provides a reagent set for chemogenomic analysis of a compound treated sample wherein the subset consists of the top-ranking 800 genes listed in Table 4, or the genes listed in Table 5. In a preferred embodiment, the invention provides a reagent set for chemogenomic analysis of a compound treated sample, wherein the reagent set is an array of polynucleotides immobilized on one or more substrates.

In another embodiment, the present invention provides a method of selecting a subset of variables out of a much larger set of multivariate data, said method comprising: (a) providing a set of multivariate data; (b) querying the data with a plurality of classification questions thereby generating a first set of classifiers comprising variables; (c) ranking each variable according to its contribution across all classifiers; and (d) selecting a subset of variables based on the ranking; whereby the subset of variables produced is sufficient to generate a second set of classifiers that perform substantially the same as or better than the first set of classifiers.

In one embodiment, the method of selecting a subset of variables out of a much larger set of multivariate data is carried out wherein the classifiers are linear classifiers reducible to weighted gene lists. In a preferred embodiment, the weighted gene lists are combined and subsets of genes of increasing size are chosen from the lists of all genes ever appearing (non-zero weighted) in any signature. In another embodiment, only those weighted gene lists forming non-redundant signatures are combined. In preferred embodiments, the method is carried out wherein gene choice is based on the sum of weights, the sum of absolute value of weights, or the sum of impacts of that gene across all signatures. Impact for a gene in a signature is defined as the product of the weight by the average expression of that gene in the class of interest. A positive weight multiplied by an average upregulation as well as a negative weight multiplied by an average downregulation, both result in a positive impact.

In other embodiments, the method of selecting a subset of variables out of a much larger set of multivariate data is carried out wherein said first set of classifiers is generated according a set of maximally diverse non-redundant questions. In some embodiments the question redundancy is determined using the fingerprint of the resulting signatures against a set of reference treatments. In another embodiment, the fingerprint of the resulting signatures may be assessed using a hierarchical clustering method selected from the group consisting of: UPGMA, WPGMA and others. Clustering methods can use a variety of distance metrics such as Pearson's correlation coefficient or Euclidean distance metric. In a preferred embodiment of the present method, the classifiers are generated using support vector machines (SVM) and the SVM algorithm used is selected from the group consisting of: SPLP, SPLR, SPMPM, ROBLP, ROBLR, and ROBMPM.

In an alternative embodiment, the resulting reduced subsets of variables generated by the method are validated as sufficient for classification tasks by a method wherein subsets of increasing size are selected and each used as input to re-compute and cross-validate the same set of non redundant classifiers used to generate the subset.

In an alternative embodiment, the invention provides a computer program product for selecting a subset of variables from a multivariate database comprising: (1) computer code for querying the multivariate database with a plurality of classification questions thereby generating a first set of classifiers comprising variables; (2) computer code for ranking each variable according to its contribution across all classifiers; and (3) computer code for selecting a subset of variables based on ranking; wherein the variables in the subset are sufficient to generate a second set of linear classifiers that perform substantially the same as or better than the first set of linear classifiers.

In some embodiments, less than the full set of non-redundant classifiers is used to generate said reduced subsets of variables of increasing size. In some embodiments, each subset of increasing size is used as input to re-compute and cross-validate the retained portion of the classifiers (e.g. the remaining 40%, 30%, 20%, 10% or less). In this embodiment, the method of validation is carried out wherein said subset achieves a substantial portion (e.g. >80%, >90% or more >95%) of the average performance, or even better than (e.g. >100%) the average performance achieved by all variables for generating valid classifiers capable of answering the retained questions. Such a reduced subset of variables is referred to as a “sufficient” set because it may be used to generate classifiers capable of answering the full set of classification questions with a performance achieving 80%, 90%, 95% or greater than 100% of the classification performance achievable when full set of variables is used to generate the same set of classifiers.

In the specific context of biological questions, the present invention provides a method for selecting a subset of biological molecules capable of answering classification questions originally addressed to a much larger multivariate set of biological data. This subset of molecules is highly-responsive to classification questions addressed to it because, although smaller than the full set, it is information rich.

In other preferred embodiments, this method may be carried out wherein the set of multivariate data was obtained from a polynucleotide array or a proteomic experiment. In addition the present method may be carried out with multivariate data from an array or proteomic experiment wherein the experiment comprises compound-treated samples.

In preferred embodiments, the variables in the reduced subset are molecules representing genes (e.g. nucleic acids, peptides or proteins), and the multivariate data is from array experiments. In this embodiment, the reduced subset of information rich genes may be used to generate classifiers (i.e. signatures) comprising short weighted lists of genes “sufficient” to answer specific diagnostic questions. A reduced subset of high-impact, responsive genes may be used to classify new samples and provide a plurality of different signatures each capable of answering a different diagnostic question. Moreover, the subset of high-impact, responsive genes provided by the method of the present invention is “universal” in that it may be used to answer novel classification questions (i.e. provide novel diagnostic assays) that were not used to originally generate the subset.

The present invention provides a method to identify a reduced subset of genes or proteins that is both sufficient and necessary to answer a wide variety of classification questions useful for developing toxicological, or pharmacological assays, or diagnostics. The method of the invention provides gene subsets that “universal” (i.e. are capable of answering novel questions not part of the initial process of selecting the gene subset).

In another embodiment of the present invention, the reduced subsets of variables may be represented by molecules (e.g. nucleic acids, peptides, etc.) in a diagnostic assay format. In one preferred embodiment, the gene subset may be represented by an array of different polynucleotides or peptides immobilized on one or more solid substrates. In one embodiment, an array of polynucleotides comprising a “universal” gene subset is immobilized on single solid substrate to form a “universal” gene chip capable of answering classification questions.

The present invention also provides an information rich subset of variables that exhibits specific characteristics with respect to the ability to classify data. In one embodiment, the invention provides a subset of variables comprising less than 10 percent of the variables in a full set of multivariate data wherein the performance of the subset of variables in answering classification questions is at least 85 percent of the performance of the full set of multivariate data in answering the same classification questions.

In one embodiment, the invention provides a subset of variables comprising those variables with the highest ranking 10 percent of impact factors across the full set of classifiers derived from a set of multivariate data.

In one embodiment, the invention provides a subset of variables comprising the variables whose removal from a set of multivariate data results in a depleted subset of variables that are unable to answer classification questions with an average logodds ratio greater than 4.8.

In one embodiment, the invention provides a subset of variables representative of a plurality of classifiers, wherein the subset is predictive of classifiers not used to generate the subset. In preferred embodiments, the variables are genes and the classifiers are chemogenomic classifiers.

In another embodiment, the invention provides an apparatus for classifying a sample comprising at least one detector for each member of a subset of variables comprising less than 10 percent of the variables in a full set of multivariate data wherein the performance of the subset of variables in answering classification questions is at least 85 percent of the performance of the full set of multivariate data in answering the same classification questions. In a preferred embodiment, the detectors are polynucleotides or polypeptides.

In another embodiment, the present invention provides a subset of “universal” genes for chemogenomic analysis of compound treated liver tissue. This subset consists of the top-ranking 800 genes listed in Table 4. Re-computing and cross validating the 116 distinct liver tissue signatures using this universal set of 800 genes as input results in a set of 116 new valid signatures that function as well as, or better than, the original 116 signatures but require the use of only 800 genes. In another embodiment, the “universal” subset includes only those genes that encode secreted proteins listed in Table 5.

### BRIEF DESCRIPTION OF THE DRAWINGS

FIG. 1 depicts (A) Hierarchical clustering of correlations between 311 drug treatments and each of 439 gene signatures; (B) depicts an enlarged portion (marked by a blue dotted box in the upper left corner of A) of the clustering plot described in FIG. 3A. The names of signatures associated with three of the clusters present in this enlargement are shown on the right.

FIG. 2 depicts an illustrative portion of the impact table that includes each of 3421 genes in the 116 non-redundant liver signatures. Impact of a gene in a signature is defined as the product of the weigh of the gene in the signature times the average gene expression log ratio for all members of the positive class-of interest for that same signature. The “upper left” portion of the table is shown. The entire list of the 3421 genes and its associated impact factor based ranking is provided in Table 4 (included as the ASCII formatted file named “Table_{—}4.txt” included on the accompanying CD, which is hereby incorporated by reference herein).

FIG. 3 depicts (A) Validation of “sufficient” sets of various sizes. Demonstration that after selection of a subset of genes, large portions of the maximum performance are retained by various size gene lists. Performance is expressed as the average test logodds ratios for 116 three-fold cross validated signatures (left panel); performance is also expressed as percent of the maximum achieved when all genes are submitted to the classification algorithm (right panel). (B) Validation of the “necessary set. The effect of removing the 3421 high impact gene (the “necessary” set) or an equal number of random genes is shown.

FIG. 4 depicts (A) Using the signature impact choice method to identify a small set of genes that contain all of the information necessary to fully classify the dataset. The plot shows the average logodds ratio (LOR) versus number of genes, chosen using the impact choice method or randomly, in various sized subsets derived from the original set of 8565 genes. The change in position between the two stars illustrates the significant drop off in performance of the remaining 5144 genes after either the high impact “necessary” set of 3421 genes is removed (five-pointed star), or a random set of 3421 genes is removed (four-pointed star) from the full data set. The data in FIG. 4 (A) are a graphic representation of the data presented in FIG. 3. (B) A plot of performance, for answering novel classification questions (in terms of average LOR), for various sized reduced subsets of genes. Each curve corresponds to a different gene choice method. The random and standard deviation based curves are shown for reference. In the curve labeled “Training on 116 signatures. Testing on same 116,” the genes were chosen based on their impact across all signatures. In the last three curves (labeled “ . . . Test on remaining 10” or “ . . . Test on remaining 58” or “ . . . Test on remaining 87”) the choice of genes is based on decreasing the number of signatures and the performance of the gene set is assessed on the remaining signatures.

### DETAILED DESCRIPTION OF THE INVENTION

I. Overview

The present invention provides a method for identifying relevant end-points and preparing small, high-throughput devices and assays useful for answering the same chemogenomic classification questions that are typically performed on much larger (and costlier) DNA microarrays. These techniques, however, are not limited to chemogenomic analysis applications. They also may be applied generally for preparing high-throughput measurement devices based on the ability of the disclosed methods to reduce large multivariate datasets to small subsets of information rich variables. For example, methods of metabolite analysis and proteomic analysis, such as: single and multiple mass spectrometry (MS, and MS/MS); liquid chromatography followed by mass spectrometry (LC/MS); electrophoresis followed by mass spectrometry (CE/MS or gel-electrophoresis/MS); and other protein analysis methods capable of measuring a large number of different analytes simultaneously. Each of these methods requires relatively little optimization for any individual analyte. These methods also produce large quantities of data that can be burdensome unless reduced to simpler assays by identification of the relevant end-points. This reduction allows simpler devices compatible with low cost high throughput multi-analyte measurement.

In a more general aspect, the present invention provides a method that allows one to select a reduced subset of information rich, responsive genes capable of answering classification questions regarding a dataset with a level of performance as good as or better than the complete gene set. Furthermore, this method may be used broadly to provide a subset of variables from any multivariate dataset wherein this subset of variables is capable of answering novel classification questions regarding the multivariate dataset. Consequently, present invention makes it possible to develop novel toxicology or pharmacology signatures, or diagnostic assays based on the analysis of greatly reduced datasets.

Significantly, the methods of the present invention provide subsets of variables capable of answering novel classification questions with a performance similar or superior to that obtained when using all the variables of the full multivariate dataset. Because they can answer novel classification questions these subsets are considered to have “universal” value. The “universal” aspect of the reduced “sufficient” subsets of the invention is significant because it allows a researcher to use a reduced subset for new classification tasks without further validation studies. Subsets whose performance approaches or surpasses that of the full set of all variables are deemed “sufficient” sets because they contain all the information present in the full set of variables. The largest “sufficient” subset defines a “necessary” set. The “necessary” set is a subset of variables whose removal from the full set of all variables results in a “depleted” set whose performance in classification tasks does not rise above a defined minimum level.

In one particularly significant application, a reduced subset of “universal” variables derived from a multivariate dataset may be incorporated into a device capable of measuring changes in the sample components corresponding to the variables. Such a measurement device may be used to answer novel classification questions by detecting changes in a subset of the “universal” variables known to correspond to a specific signature.

II. Definitions

“Multivariate dataset” as used herein, refers to any dataset comprising a plurality of different variables including but not limited to chemogenomic datasets comprising logratios from differential gene expression experiments, such as those carried out on polynucleotide microarrays, or multiple protein binding affinities measured using a protein chip. Other examples of multivariate data include assemblies of data from a plurality of standard toxicological or pharmacological assays (e.g. blood analytes measured using enzymatic assays, antibody based ELISA or other detection techniques).

“Variable” as used herein, refers to any value that may vary. For example, variables may include relative or absolute amounts of biological molecules, such as mRNA or proteins, or other biological metabolites. Variables may also include dosing amounts of test compounds.

“Classifier” as used herein, refers to a function of a set of variables that is capable of answering a classification question. A “classification question” may be of any type susceptible to yielding a yes or no answer (e.g. “Is the unknown a member of the class or does it belong with everything else outside the class?”). “Linear classifiers” refers to classifiers comprising a first order function of a set of variables, for example, a summation of a weighted set of gene expression logratios. A valid classifier is defined as a classifier capable of achieving a performance for its classification task at or above a selected threshold value. For example, a log odds ratio≧4.00 represents a preferred threshold of the present invention. Higher or lower threshold values may be selected depending of the specific classification task.

“Signature” as used herein, refers to a combination of variables, weighting factors, and other constants that provides a unique value or function capable of answering a classification question. A signature may include as few as one variable. Signatures include but are not limited to linear classifiers comprising sums of the product of gene expression logratios by weighting factors and a bias term.

“Weighting factor” (or “weight”) as used herein, refers to a value used by an algorithm in combination with a variable in order to adjust the contribution of the variable.

“Impact factor” or “Impact” as used herein in the context of classifiers or signatures refers to the product of the weighting factor by the average value of the variable of interest. For example, where gene expression logratios are the variables, the product of the gene's weighting factor and the gene's measured expression log₁₀ ratio yields the gene's impact. The sum of the impacts of all of the variables (e.g. genes) in a set yields the “total impact” for that set.

“Scalar product” (or “Signature score”) as used herein refers to the sum of impacts for all genes in a signature less the bias for that signature. A positive scalar product for a sample indicates that it is positive for (i.e., a member of) the classification that is determined by the classifier or signature.

“Sufficient set” as used herein is a set of variables (e.g. genes, weights, bias factors) whose cross-validated performance for answering a specific classification question is greater than an arbitrary threshold (e.g. a log odds ratio≧4.0).

“Necessary set” as used herein is a set of variables whose removal from the full set of all variables results in a depleted set whose performance for answering a specific classification question does not rise above an arbitrarily defined minimum level (e.g. log odds ratio≧4.00).

“Log odds ratio” or “LOR” is used herein to summarize the performance of classifiers or signatures. LOR is defined generally as the natural log of the ratio of the odds of predicting a subject to be positive when it is positive, versus the odds of predicting a subject to be positive when it is negative. LOR is estimated herein using a set of training or test cross-validation partitions according to the following equation,

$$LOR = ln \frac{( {\sum }_{i = 1}^{c} {TP}_{i} + 0.5 ) * ( {\sum }_{i = 1}^{c} {TN}_{i} + 0.5 )}{( {\sum }_{i = 1}^{c} {FP}_{i} + 0.5 ) * ( {\sum }_{i = 1}^{c} {FN}_{i} + 0.5 )}$$

where c (typically c=40 as described herein) equals the number of partitions, and TPᵢ, TNᵢ, FPᵢ, and FNᵢ represent the number of true positive, true negative, false positive, and false negative occurrences in the test cases of the i^{th} partition, respectively.

“Array” as used herein, refers to a set of different biological molecules (e.g. polynucleotides, peptides, carbohydrates, etc.). An array may be immobilized in or on one or more solid substrates (e.g., glass slides, beads, or gels) or may be a collection of different molecules in solution (e.g., a set of PCR primers). An array may include a plurality of biological polymers of a single class (e.g. polynucleotides) or a mixture of different classes of biopolymers (e.g. an array including both proteins and nucleic acids immobilized on a single substrate).

“Array data” as used herein refers to any set of constants and/or variables that may be observed, measured or otherwise derived from an experiment using an array, including but not limited to: fluorescence (or other signaling moiety) intensity ratios, binding affinities, hybridization stringency, temperature, buffer concentrations.

“Proteomic data” as used herein refers to any set of constants and/or variables that may be observed, measured or otherwise derived from an experiment involving a plurality of mRNA translation products (e.g. proteins, peptides, etc) and/or small molecular weight metabolites or exhaled gases associated with these translation products.

III. Multivariate Datasets

a. Various Useful Multivariate Data Types

The present invention may be used with a wide range of multivariate data types to generate reduced subsets of highly informative variables. These reduced subsets of variables may be used to prepare lower cost, higher throughput assays and associated devices. A preferred application of the present invention is in the analysis of data generated by high-throughput biological assays such as DNA array experiments, or proteomic assays. For example, as larger multivariate data sets are assembled for large sets of molecules (e.g. small or large chemical compounds) the present method may be applied to these reduce these datasets and allow the facile generation of linear classifiers. The large datasets may include any sort of molecular characterization information including, e.g. spectroscopic data (e.g. UV-Vis, NMR, IR, mass spectrometry, etc.), structural data (e.g. three-dimensional coordinates) and functional data (e.g. activity assays, binding assays). The reduced subsets of data produced by using the present invention on such a dataset could then be applied to generate linear classifiers for this molecular dataset that would be useful in a multitude of analytical contexts, including the development and manufacture of derivative detection devices.

In another example, one may imagine reducing a large multivariate dataset of human metabolite levels to a small subset that could be used to generate a simplified detection device for various different ingested toxins. The present invention would provide a reduced subset of metabolite levels that could be used to create a universal poisoning detector used by emergency medical personnel.

Generally, the present invention will be useful wherever reduction of large multivariate datasets allows one to simplify data classification. One of ordinary skill will recognize that the methods of the present invention may be applied to multivariate data in areas outside of biotechnology, chemistry, pharmaceutical or the life sciences. For example, the present invention may be used in physical science applications such as climate prediction, or oceanography, where it is essential to reduce large data sets and prepare simple signatures capable of being used for detection.

Large dataset classification problems are common in the finance industry (e.g. banks, insurance companies, stock brokers, etc.) A typical finance industry classification question is whether to grant a new insurance policy (or home mortgage) versus not. The variables to consider are any information available on the prospective customer or, in the case of stock, any information on the specific company or even the general state of the market. The finance industry equivalent to the above described “gene signatures” would be financial signatures for a specific decision. The present invention would identify a reduced set of variables worth collecting from customers that could be used to derive financial decision for all questions of a given type.

b. Construction of a Multivariate Dataset

As discussed above, the data reduction method of the present invention may be used to derive (i.e. “mine”) reduced subsets of responsive variables from any multivariate data set. In preferred embodiments the dataset comprises chemogenomic data.

For example, the data may correspond to treatments of organisms (e.g. cells, worms, frogs, mice, rats, primates, or humans etc.) with chemical compounds at varying dosages and times followed by gene expression profiling of the organisms transcriptome (e.g. measuring mRNA levels) or proteome (e.g. measuring protein levels). In the case of multicellular organisms (e.g. mammals) the expression profiling may be carried out on various tissues of interest (e.g. liver, kidney, marrow, spleen, heart, brain, intestine). In addition to the expression profile data, the chemogenomic dataset may include additional data types such as data from classic biochemistry assays carried out on the organisms, and/or tissue of interest. Other data included in a large multivariate dataset may include histopathology, and pharmacology assays, and structural data for the chemical compounds of interest.

One example of a chemogenomic multivariate dataset based on DNA microarray expression profiling data is described in Published U.S. Appl. No. 2005/0060102 A1 (entitled “Interactive Correlation of Compound Information and Genomic Information”) which is hereby incorporated by reference for all purposes.

Microarrays are well known in the art and consist of a substrate to which probes that correspond in sequence to genes or gene products (e.g., cDNAs, mRNAs, cRNAs, polypeptides, and fragments thereof), can be specifically hybridized or bound at a known position. The microarray is an array of reagents capable of detecting genes (e.g., a DNA or protein) immobilized on a single solid support in which each position represents a discrete site for detecting a specific gene. Typically, the microarray includes sites with reagents capable of detecting many or all of the genes in an organism's genome.

As disclosed above, a treatment may include but is not limited to the exposure of a biological sample or organism (e.g. a rat) to a drug candidate, the introduction of an exogenous gene into a biological sample, the deletion of a gene from the biological sample, or changes in the culture conditions of the biological sample. Responsive to a treatment, a gene corresponding to a microarray site may, to varying degrees, be (a) upregulated, in which more mRNA corresponding to that gene may be present, (b) downregulated, in which less mRNA corresponding to that gene may be present, or (c) unchanged. The amount of upregulation or downregulation for a particular matrix location is made capable of machine measurement using known methods which cause photons of a first wavelength (e.g., green) to be emitted for upregulated genes and photons of a second wavelength (e.g., red) to be emitted for downregulated genes.

After treatment and appropriate processing of the microarray, the photon emissions are scanned into numerical form, and an image of the entire microarray is stored in the form of an image representation such as a color JPEG format. The presence and degree of upregulation or downregulation of the gene at each microarray site represents, for the perturbation imposed on that site, the relevant output data for that experimental run or “scan.”

The methods for reducing datasets disclosed herein are broadly applicable to other gene and protein expression data. For example, in addition to microarray data, biological response data including gene expression level data generated from serial analysis of gene expression (SAGE, supra) (Velculescu et al., 1995, Science, 270:484) and related technologies are within the scope of the multivariate data suitable for analysis according to the method of the invention. Other methods of generating biological response signals suitable for the preferred embodiments include, but are not limited to: traditional Northern and Southern blot analysis; antibody studies; chemiluminescence studies based on reporter genes such as luciferase or green fluorescent protein; Lynx; READS (GeneLogic); and methods similar to those disclosed in U.S. Pat. No. 5,569,588, which is hereby incorporated by reference herein in its entirety.

In another preferred embodiment, the large multivariate dataset may include genotyping (e.g. single-nucleotide polymorphism) data. The present invention may be used to reduce large datasets of genotype information to small subsets of specific high-impact SNPs that are most useful for a diagnostic or pharmacogenomic assay.

Generally, the more comprehensive the original large multivariate dataset, the more robust and useful will be the reduced subset of variables derived using the method of the invention. For example, in the case of a chemogenomic database, the ability of a reduced subset of genes to generate a new classifier (i.e., signature) will be limited where the pertinent classification question requires a gene (or pathway of genes) that was never sampled in constructing the original large dataset.

The method of generating a multivariate dataset which may be reduced according to the present invention is aided by the use of relational database systems for storing and retrieving large amounts of data. The advent of high-speed wide area networks and the Internet, together with the client/server based model of relational database management systems, is particularly well-suited for meaningfully analyzing large amounts of multivariate data given the appropriate hardware and software computing tools. Computerized analysis tools are particularly useful in experimental environments involving biological response signals. For example a large chemogenomic dataset may be constructed as described in Published U.S. Appl. No. 2005/0060102 A1 (entitled “Interactive Correlation of Compound Information and Genomic Information”) which is hereby incorporated by reference for all purposes.

Generally, multivariate data may be obtained and/or gathered using typical biological response signal matrices, that is, physical matrices of biological material that transmit machine-readable signals corresponding to biological content or activity at each site in the matrix. In these systems, responses to biological or environmental stimuli may be measured and analyzed in a large-scale fashion through computer-based scanning of the machine-readable signals, e.g. photons or electrical signals, into numerical matrices, and through the storage of the numerical data into relational databases.

IV. Classification Questions, Linear Classifiers and Redundancy

a. Comprehensive Data Mining of a Large Multivariate Dataset with Classification Questions

The initial questions used to classify (i.e. the classification questions) a large multivariate dataset may be of any type susceptible to yielding a yes or no answer. The general form of such questions is: “Is the unknown a member of the class or does it belong with everything else outside the class?” For example, in the area of chemogenomic datasets, classification questions may include “mode-of-action” questions such as “All treatments with drugs belonging to a particular structural class versus the rest of the treatments” or pathology questions such as “All treatments resulting in a measurable pathology versus all other treatments.” In the specific case of chemogenomic datasets based on gene expression, it is preferred that the classification questions are further categorized based on the tissue source of the gene expression data. Similarly, it may be helpful to sub-divide other types of large data sets so that specific classification questions are limited to particular subsets of data. Typically, the significance of sub-dividing data within large datasets become apparent upon initial attempts to classify the complete dataset. A principle component analysis and/or a t-ranked discrimination metric treatment of the complete dataset may be used to identify the subdivisions in a large dataset (see e.g., US 2003/0180808 A1 and US 2004/0259764 A1, each of which is hereby incorporated by reference herein.)

In order to prepare reduced subsets variables that exhibit the most robust performance relative to the full dataset, it is important to scan the complete classification-space. To do this, one must query the original dataset with all classification questions that the dataset can conceivably answer in a systematic fashion. That is, an attempt should be made to generate a classifier for every single class definable in the database. In order to identify valid classifiers, a threshold performance is set for an answer to the particular classification question. In one preferred embodiment, the classifier threshold performance is set as logodds ratio greater than 4.00 (i.e. LOR>4). However, higher or lower thresholds may be used depending on the particular dataset and the desired properties of the classifiers so obtained. Of course many queries of the dataset with a classification will not generate a valid classifier.

b. Algorithms for Generating Valid Classifiers

Comprehensive dataset classification may be carried out manually, that is by evaluating the dataset by eye and classifying the data accordingly. However, because the dataset may involve tens of thousands (or more) individual variables, more typically, the querying of the full dataset with the classification questions is carried out in a computer employing any of the well-known data classification algorithms.

In preferred embodiments, algorithms may be used that generate linear classifiers. In particularly preferred embodiments the algorithm is selected from the group consisting of: SPLP, SPLR and SPMPM. These algorithms are based respectively on Support Vector Machines (SVM), Logistic regression (LR) and Minimax Probability Machine (MPM). They have been described in PCT Publication No. WO 2004/037200, which is hereby incorporated by reference herein in its entirety (See also, El Ghaoui, et al., “Robust classifiers with interval data” Report # UCB/CSD-03-1279, Computer Science Division (EECS), University of California, Berkeley, Calif. (2003); Brown et al., “Knowledge-based analysis of microarray gene expression data by using support vector machines,” Proc Natl Acad Sci U S A 97: 262-267 (2000)).

Generally, the sparse classification methods SPLP, SPLR, SPMPM are linear classification algorithms in that they determine the optimal hyperplane separating a positive and a negative class. This hyperplane, H can be characterized by a vectorial parameter, w (the weight vector) and a scalar parameter, b (the bias): H={x|I w^{T}x+b=0}.

For all proposed algorithms, determining the optimal hyperplane reduces to optimizing the error on the provided training data points, computed according to some loss function (e.g. the “Hinge loss,” i.e. the loss function used in 1-norm SVMs; the “LR loss;” or the “MPM loss” augmented with a 1-norm regularization on the signature, w. Regularization helps to provide a sparse, short signature. Moreover, this 1-norm penalty on the signature will be weighted by the average standard error per gene. That is, genes that have been measured with more uncertainty will be less likely to get a high weight in the signature. Consequently, the proposed algorithms lead to sparse signatures, and takes into account the average standard error information.

Mathematically, the algorithms can be described by the cost functions (shown below for SPLP, SPLR and SPMPM) that they actually minimize to determine the parameters w and b.

SPLP

$${min}_{w , b} {\sum }_{i} {e}_{i} + ρ {\sum }_{i} {σ}_{i} \vert {w}_{i} \vert s . t . {y}_{i} ( {w}^{T} {x}_{i} + b ) \geq 1 - {e}_{i}$$

$${e}_{i} \geq 0 , i = 1 , … , N$$

The first term minimizes the training set error, while the second term is the 1-norm penalty on the signature w, weighted by the average standard error information per gene given by sigma. The training set error is computed according to the so-called Hinge loss, as defined in the constraints. This loss function penalizes every data point that is closer than “1” to the separating hyperplane H, or is on the wrong side of H. Notice how the hyperparameter rho allows trade-off between training set error and sparsity of the signature w.

SPLR

$${min}_{w , b} {\sum }_{i} log ( 1 + exp ( - {y}_{i} ( {w}^{T} {x}_{i} + b ) ) ) + ρ {\sum }_{i} {σ}_{i} \vert {w}_{i} \vert$$

The first term expresses the negative log likelihood of the data (a smaller value indicating a better fit of the data), as usual in logistic regression, and the second term will give rise to a short signature, with rho determining the trade-off between both.

SPMPM

$${min}_{w} \sqrt{{w}^{T} {\hat{Γ}}_{+} w} + \sqrt{{w}^{T} {\hat{Γ}}_{-} w} + ρ {\sum }_{i} {σ}_{i} \vert {w}_{i} \vert s . t . {w}^{T} ( {\hat{x}}_{+} - {\hat{x}}_{-} ) = 1$$

Here, the first two terms, together with the constraint are related to the misclassification error, while the third term will induce sparsity, as before. The symbols with a hat are empirical estimates of the covariances and means of the positive and the negative class. Given those estimates, the misclassification error is controlled by determining w and b such that even for the worst-case distributions for the positive and negative class (which we do not exactly know here) with those means and covariances, the classifier will still perform well. More details on how this exactly relates to the previous cost function can be found in e.g. El Ghaoui et al., op. cit.

As mentioned above, classification algorithms capable of producing linear classifiers are preferred for use with the present invention. In the context of chemogenomic datasets, linear classifiers may be reduced to a series of genes and associated weighting factors. Linear classification algorithms are particularly useful with DNA array or proteomic datasets because they provide simplified gene signatures useful for answering a wide variety of questions related to biological function and pharmacological/toxicological effects associated with genes. Gene signatures are particularly useful because they are easily incorporated into wide variety of DNA- or protein-based diagnostic assays (e.g. DNA microarrays).

However, some classes of non-linear classifiers, so called kernel methods, may also be used to develop short gene lists, weights and algorithms that could also be used in diagnostic device development; while the preferred embodiment described here uses linear classification methods, specifically contemplate that non-linear methods may also be suitable.

Classifications may also be carried using principle component analysis and/or t-ranked discrimination metric algorithms as described in US 2003/0180808 A1 and US 2004/0259764 A1, each of which is hereby incorporated by reference herein).

Cross-validation of signatures may be used to insure optimal performance. Methods for cross-validation are described by PCT Publication No. WO 2004/037200, which is hereby incorporated by reference herein in its entirety. Briefly, for cross-validation of signatures, the dataset is randomly split. A training signature is derived from the training set composed of 60% of the samples and used to classify both the training set and the remaining 40% of the data, referred to here as the test set. In addition, a complete signature is derived using all the data. The performance of these signatures can be measured in terms of log odds ratio (LOR) or the error rate (ER) defined as:
LOR=ln(((TP+0.5)*(TN+0.5))/((FP+0.5)*(FN+0.5)))
and
ER=(FP+FN)/N;

where TP, TN, FP, FN, and N are true positives, true negatives, false positives, false negatives, and total number of samples to classify, respectively, summed across all the cross validation trials. The performance measures are used to characterize the complete signature, the average of the training or the average of the test signatures.

c. Producing a Set of Maximally Divergent Non Redundant Classifiers.

As mentioned above, in order to generate a more robust reduced subset of variables, it is important to query the large multivariate dataset as comprehensively as possible, that is, ask as many questions as the dataset might reasonably be expected to provide answers. One way to do this is to systematically and exhaustively probe the dataset with classification questions that span the full spectrum of classes that may exist in the dataset. However, such an exhaustive analysis offer results in querying classification questions that have the same answer and therefore generate redundant classifiers. While the presence of redundant classifiers does not prevent one from generating a useful reduced set of variables, they prevent full compression of the large multivariate dataset. Consequently, in preferred embodiments of the invention, redundant classifiers (i.e. signatures) are eliminated from initial set of classifiers generated from the large multivariate dataset using the methods disclosed herein.

Two or more signatures may be redundant or synonymous for a variety of reasons. Apparently different classification questions (i.e. class definitions) may result in identical classes and therefore identical signatures. For instance, the following two class definitions define the exact same treatments in the database: (1) all treatments with molecules structurally related to statins; and (2) all treatments with molecules having an IC₅₀<1 μM for HMGCoA reductase.

In addition, when a large dataset is queried with the same classification question using different algorithms (or even the same algorithm under slightly different conditions) different, valid signatures may be obtained. These different signatures may or may not comprise an overlapping gene set however they each can accurately identify members of the class of interest. As illustrated in Table 1, two signatures for the fibrate class of drugs were generated with the only difference being the algorithm utilized. Genes are designated by their accession number and a brief description. The weights associated with each gene are also indicated. Each signature was trained on the exact same 60% of the multivariate dataset and then cross validated on the exact same remaining 40% of the dataset. Both signatures were shown to exhibit the exact same level of performance as classifiers: two errors on the cross validation data set. The SPLP derived signature consists of 20 genes. The SPLR derived signature consists of eight genes. Only three of the genes from the SPLP signature are present in the eight gene SPLR signature.

TABLE 1
Two Signatures for the Fibrate Class of Drugs

| | Accession | Weight | Unigene name |
|---|---|---|---|
| RLPC | K03249 | 1.1572 | enoyl-Co A, hydratase/3-hydroxyacyl |
| | | | Co A dehydrogenase |
| | AW916833 | 1.0876 | hypothetical protein RMT-7 |
| | BF387347 | 0.4769 | ESTs |
| | BF282712 | 0.4634 | ESTs |
| | AF034577 | 0.3684 | pyruvate dehydrogenate kinase 4 |
| | NM_019292 | 0.3107 | carbonic anhydrase 3 |
| | AI179988 | 0.2735 | ectodermal-neural cortex (with |
| | | | BTB-like domain) |
| | AI715955 | 0.211 | Stac protein (SRC homology 3 and |
| | | | cysteine-rich domain protein) |
| | BE110695 | 0.2026 | activating transcription factor 1 |
| | J03752 | 0.0953 | microsomal glutathione S-transferase |
| | | | 1 |
| | D86580 | 0.0731 | nuclear receptor subfamily 0, group |
| | | | B, member 2 |
| | BF550426 | 0.0391 | KDEL (Lys-Asp-Glu-Leu) endoplasmic |
| | | | reticulum protein retention receptor |
| | | | 2 |
| | AA818999 | 0.0296 | muscleblind-like 2 |
| | NM_019125 | 0.0167 | probasin |
| | AF150082 | −0.0141 | translocase of inner mitochondrial |
| | | | membrane 8 (yeast) homolog A |
| | BE118425 | −0.0781 | Arsenical pump-driving ATPase |
| | NM_017136 | −0.126 | squalene epoxidase |
| | AI171367 | −0.3222 | HSPC154 protein |
| | NM_019369 | −0.637 | inter alpha-trypsin inhibitor, |
| | | | heavy chain 4 |
| | AI137259 | −0.7962 | ESTs |
| SPLR | NM_017340 | 5.3688 | acyl-coA oxidase |
| | BF282712 | 4.1052 | ESTs |
| | NM_012489 | 3.8462 | acetyl-Co A acyltransferase 1 |
| | | | (peroxisomal 3-oxoacyl-Co A |
| | | | thiolase) |
| | BF387347 | 1.767 | ESTs |
| | K03249 | 1.7524 | enoyl-Co A, hydratase/3-hydroxyacyl |
| | | | Co A dehydrogenase |
| | NM_016986 | 0.0622 | acetyl-co A dehydrogenase, medium |
| | | | chain |
| | AB026291 | −0.7456 | acetoacetyl-CoA synthetase |
| | AI454943 | −1.6738 | likely ortholog of mouse porcupine |
| | | | homolog |

One method of reducing redundancy requires making an a priori examination of the class definitions (i.e. classification questions) used to query the dataset and then eliminating those that appear likely to yield the same, or similar, answer. However, this approach requires a high level of chemical and biological knowledge and intuition and requires many fine distinctions between similar class property descriptions, several of which may be evaluated differently, even by the same reviewing scientist, on different days, depending only on the circumstances of the scientist recent experiences and thinking. Thus, these a priori best judgment examinations of the signature relationship can be quite subjective. A more desirable, objective approach to the issue of signature relationship and redundancy is described below.

In a preferred embodiment of the present invention, an empirical correlation clustering method may be used to select non-redundant signatures useful for generating a reduced subset of variables. Generally, a classifier or signature is considered non-redundant if it creates a distinct “fingerprint” when used on the complete, or a large subset of, the dataset.

It is believed that empirical correlation clustering method takes into account all sources of functional redundancy and has the advantage of quantitatively defining the redundancy threshold based on actual experimental data, and thus is not subjective.

In one aspect, the set of non-redundant classifiers itself represents a reduced set of high value classification questions. Because these questions represent the full-scope of classifications available for the dataset and if the dataset is very large and encompasses most, or all, of the possible response mechanisms available to the organism or tissue, they may be used to classify new unknown experimental data, with little or no loss of information.

V. Identifying a Reduced Subset of Information Rich Variables and Validating their Performance for Classification Tasks

It is an object of this invention to demonstrate that the information present in an initial set of signatures may be used to select subsets of information rich genes capable of generating signatures that perform comparably or better than the initial set.

A. Calculating and Ranking Impact Factors

Once a set of classifiers or signatures is derived for a large multivariate dataset, the data may be re-assembled as a single table of variables versus classifiers. This table may then be used to identify “high information content,” “highly responsive,” and/or “information rich” variables that are most useful for preparing a high throughput diagnostic device from a reduced subset.

Generally, identification of information enriched variables involves deconstructing each of the classifier spanning the whole dataset into its constituent variables. For example, in the case of a chemogenomic dataset, the linear classifiers may be deconstructed into a list of the genes and associated weighting factors comprising the classifier. The weighting factors associated with each variable in each linear classifier may then be inserted in the cells of a table (i.e. matrix) of variables versus classifiers. The weighting factors for each variable across all signatures may then be summed to calculate an overall contribution for each variable. Alternatively, an “impact factor” may be calculated by summing the product of the weighting factors for each variable and the average value of that variable, usually restricted to the average value of the variable in positive class for the classification question. Typically a threshold level is set for assignment of a non-zero weighting factor. Depending on this threshold level, the resulting impact table may be more or less sparse (i.e. populated with few non-zero values).

A cursory examination of the impact table should indicate the extent to which the full subset may be reduced. If only a few variables appear to have non-zero values in many of the classifiers, it is likely that the dataset can be reduced to a much smaller yet high-performing subset of variables.

The total impact factor calculated for each variable across the complete set of classifiers may be used to rank the variables for selection as part of the reduced subset. Generally, the variables selected for the reduced subset may be chosen based on the rank of its summed impacts across all classifiers. However, alternative methods of selection may be used with the present invention. For example, selection may be based directly on a sum of weighting factors or a sum of absolute values of weighting factors. This minor modification in the overall dataset reduction method may provide an even smaller and better performing reduced sets.

The selection of the variables for the reduced subset may be based on the rank of the variables impact factor relative to those for all other variables in the full dataset. In some embodiments, the cut-off for inclusion of a variable in the reduced subset is determined based on the application intended for the reduced subset. Different diagnostic devices may accommodate different numbers of genes. In some embodiments, the ranking cut-off threshold may be set so that less than 50%, 25%, 10% or even less than 5% of the variables from the full dataset are included in the reduced subset.

Alternatively, or in addition, a number of different sized subsets may be selected and then empirically validated for performance in answering classification questions relative to the full dataset. In one typical embodiment, a minimal logodds ratio of 4.8 is set and different sized reduced subsets are validated for ability to generate the set of non-redundant classifiers. Those subsets that perform with a logodds ratio<4.8 are disregarded in favor of the reduced subsets that perform better than LOR=4.8. Of course, higher or lower LOR standards may be used in selecting the subset. For example, subsets performing with LOR >2.5, 3.0, 4.0, 4.25, 4.5, 4.75, 5.00, 5.25 or 5.50 may be selected. In a preferred embodiment, the subset with the fewest variables that still performs with a LOR greater than desired level is selected.

Regardless of the criteria used to select the cut-off for the variables included in the reduced subset, the method of the present invention allows one to optimize subset size for the specific analytical purpose desired. For example, in developing a DNA array device for rapid toxicology screening of mRNA from treated rat liver samples, the size of the selected gene subset may be determined based on the desired throughput, cost, the total number of genes needed, or the total number of samples to be analyzed.

The present invention thus opens the door to varying levels of diagnostic devices each with its own “sweet spot” defined in terms of the classification performance parameters relative to that of a much more expensive device capable of monitoring a much larger complete set of variables.

B. Validating Reduced Subset Performance

Cross-validation experiments may be used to confirm that the average performance of the highly reduced subsets of variables is as good as, or better than, the original large dataset for classifying data. Furthermore, cross-validation experiments may be used to determine whether a subset is “sufficient” to perform as well as the complete set.

Cross validation may be carried out by querying the selected subset with the complete set of classification questions in order to generate a complete set of classifiers. The performance of these subset-derived classifiers may then be used to classify the original full dataset. The performance of the subset-derived classifiers may be measured in terms of a LOR that may then be compared to the LOR for the same task carried out by the original set of classifiers derived from the full dataset. In addition, comparison may be made between subsets selected according to the method of the present invention and subsets of identical size selected randomly from the complete set of variables.

The preferred subsets made by the method of the present invention generate classifiers that perform at least 85%, 90%, or 95% as well as those generated by the complete dataset. Depending on the amount of reduction of the subset, the performance of the derived classifiers may be substantially the same as or even better than the classifiers derived from the full set.

Thus, the method of the present invention allows one to use the information present in the initial set of signatures (derived from the full dataset) and ultimately select a subset of variables that provides an even better, or at least nearly equal, performing set of signatures.

A reduced subset made by the method of the present invention is not necessarily unique in its ability to classify the complete dataset. Slight variations in the method and criteria used to select the subset may yield a subset that does not completely overlap yet has comparable performance. For example, when weighting factors alone, rather than a product impact factor is used to rank variables the resulting subset only partially overlaps the impact-based subset but may produce similar results in terms of performance.

C. Validating “Necessary” Subsets.

Empirically, such a “necessary” subset may be defined as the list of variables, N, selected from the list of all variables present in the full dataset, A, such that the performance of the remaining variables, R (where R=A-N), never rises above some minimal threshold. This threshold may be arbitrary and may be used to define how “necessary” a particular subset is. One possible choice for a threshold level that may be used is the level of performance achieved by the smallest “sufficient” subset identified according to the methods described above (e.g. a subset exhibiting a LOR >4.8).

D. Validating Performance of Reduced Subsets for Answering Novel Classification Questions

A further significant question is whether the reduced subsets made using the method of the present invention are capable of generating novel classifiers. Novel classifiers would include signatures generated in answer to queries not posed to the complete dataset, and queries distinct from those asked during the compilation of the non-redundant signature set. A simulation involving cross-validation may be performed in order to answer this question.

In a preferred embodiment, a “split-sample” cross validation procedure may be used. Generally, this method involves a random subset of some number, N out of the original number of M classifiers originally generated from the comprehensive classification of multivariate dataset. The subset of classifiers, N, may then be used to generate subsets of variables of various size using, for example, the sum of weights or the sum of impact method described above in section V.A. Each of the variable subset are then used as input to generate the remaining (M-N) classifiers. The performance of the variable subset may be defined as the average of the test LOR for the remaining (M-N) signatures so generated. This procedure is then repeated systematically for a total of at least ten different splits N/(M-N) of the M classifiers.

This split sample procedure may be carried out for a plurality of different size subsets. A plot of results for varying sized subsets may be used to reach the conclusion that a reduced subsets made by the method described of the present invention has “universal” value; that is, it performs equally well on classification tasks that were, or were not, involved in deriving the variables in the subset.

VI. Preparation of Diagnostic Assays and Devices Using Reduced Subsets

As described above, a large dataset of variables may be reduced substantially and still perform as well or better in answering classification problems. One product of this data reduction method is the ability to produce cheaper, higher throughput diagnostic assays that include a selected subset consisting of less than 50%, 40%, 30%, 20%, 10%, or even less than 5% of the analyte probes present in a larger assay and still achieve the same level of performance for sample classification tasks.

Furthermore, the above-described cross-validation experiments demonstrate that reduced subsets of variables (e.g., genes) from a large multivariate dataset may be used to answer previously unasked classification questions with a minimal loss in performance relative to using the complete dataset. Consequently, the present invention provides small subsets of variables sufficient to form a reduced size, inexpensive “universal” diagnostic assay or device. In this context, the term “universal” is not without limitation. The spectrum of classification questions that may be answered using a reduced subset without a significant loss of performance should fall within the general scope of questions answered by the set of non-redundant classifiers used to generate the subset. Performance below a standard metric thus constitutes a boundary for the universality concept (e.g., inability to produce a valid signature for the novel classification question). For example, in the case of the chemogenomic dataset described in Example 1, which comprises gene expression changes in liver tissue caused by compound treatments, the scope of novel classification questions should be limited to effects in liver observable using a DNA microarray of the 8565 genes. Thus, if a new drug-induced rat liver pathology is identified (e.g. a previously unreported finding of “blue liver”), it should be possible using a reduced subset of genes made according to the present invention to generate a valid signature for this novel pathology. Because there is no data in the existing chemogenomic dataset related to this novel pathology it will be necessary to perform new gene expression experiments, however these new experiments need only be performed on an inexpensive DNA array featuring a greatly reduced reagent set (e.g., 800 or fewer) of polynucleotides or polypeptides capable of detecting the high impact genes in the reduced subset.

In some cases it may be desirable to use a reduced subset of genes on an assay or device platform different than the one used to generate the original dataset from the subset is derived. Although the genes in the reduced subset need not change, it may be necessary to optimize or recalibrate the signatures for the new platform. Recalibration to a new platform requires running new chemogenomic assays on that platform and re-generating the signatures. Conducting a new series of chemogenomic re-calibration experiments can be costly, time consuming and therefore offset some of the efficiencies gained by using a reduced subset of genes. However, as is shown in Example 6, the data regeneration process may be greatly abbreviated and still result in a set of signatures capable of performing at a level as good as those derived based on a much larger dataset. Key to abbreviating the recalibration process is to use of a method for “label trimming” to reduce the number of compound treatment experiments that need to be conducted on the new platform. Label trimming generally involves eliminating those compound treatments that contribute less significantly to the definition of the set of non-redundant signatures used to generate the reduced subset of genes. Three methods of label trimming are described in Example 6 below. Using signature re-calibration, any of the reduced subset of highly informative genes may be adapted to a new diagnostic assay or device according to the methods described herein.

A preferred platform that may be built using the present invention is a “universal” DNA microarray or gene chip. Once a reagent set based on a reduced subset of genes derived according to the present invention, a DNA microarray may be constructed using any of the well-known techniques by selecting only those genes found in a “sufficient” reduced subset. Such a universal microarray can be much smaller (e.g., only about 100-800 probes instead of 10,000) and consequently, much simpler and cheaper to manufacture and use. Despite its reduced complexity, the universal DNA microarray is still capable of carrying out the full range of chemogenomic classification tasks. Thus, large-scale chemogenomic studies may be carried out with newly developed compound treatments, while using greatly simplified and much cheaper universal gene chips featuring less than about 800, 700, 600, 500, 400, 300, 200, or even 100 polynucleotides capable of detecting genes in a reduced subset derived from a much larger chemogenomic dataset.

In addition to including a small set of probes, each of which is capable of detecting at least one highly informative gene from a reduced subset, in some embodiments, the universal gene chip may include additional sets of probes, not from a reduced subset, but also capable of detecting genes relevant to a specific pharmacological or toxicological classification question.

A variety of microarray formats and platforms are well-known in the art and may be used with the methods and reduced subsets of genes produced by the present invention. In one preferred embodiment, photolithographic or micromirror methods may be used to spatially direct light-induced chemical modifications of spacer units or functional groups resulting in attachment oligonucleotide probes at specific localized regions on the surface of the substrate. Light-directed methods of controlling reactivity and immobilizing chemical compounds on solid substrates are well-known in the art and described in U.S. Pat. Nos. 4,562,157, 5,143,854, 5,556,961, 5,968,740, and 6,153,744, and PCT publication WO 99/42813, each of which is hereby incorporated by reference herein.

Alternatively, a plurality of molecules (e.g., polynucleotides, or polypeptides such as monoclonal antibodies) may attached to a single substrate by precise deposition of chemical reagents. For example, methods for achieving high spatial resolution in depositing small volumes of a liquid reagent on a solid substrate are disclosed in U.S. Pat. Nos. 5,474,796 and 5,807,522, both of which are hereby incorporated by reference herein.

It should also be noted that the term “universal” does not imply that a single diagnostic assay or device would satisfy all needs. For example, in the case of chemogenomic analysis of compound-treated rats, it may be desirable to prepare different arrays based on different reagent sets for each tissue. Alternatively, a single substrate (or set of substrates, e.g., beads) may be produced with several different small arrays of 100 or so probes localized in different areas on the surface of the substrate. Each of the different arrays may represent a sufficient subset of genes for a particular tissue. In addition, it may be desirable to investigate classification questions of a different nature in the same tissue using several different “universal” arrays of relatively small gene sets. In another alternative embodiment, microarrays with greatly reduced probe numbers may be desirable for initial exploratory investigation (e.g. classifying drug treated rats). In addition, DNA arrays of varying size (number of genes), each adapted to a specific follow-up technology may also be created.

The diagnostic assays and devices prepared using the reduced subsets described by the present invention are universal in the sense that they are “sufficient” to answer questions that were not part of the original subset selection process. The scope of classifiers for which they are useful however may be limited depending on the scope of the original questions used to query the dataset; for example the above described universal gene set might not be useful in applications studying tissue or organ development.

Although DNA microarrays represent a preferred embodiment, the methodology described herein may be applied to other types of datasets. Indeed, any of the methods well-known in the art for measuring gene expression, at either the transcript level or the protein level, may be used as a platform for a reduced subset of genes for chemogenomic analysis. Methods for preparing the particular reagent sets that may be used to detect the reduced subset genes are well-known to the skilled artisan. For example, proteomics assay techniques, where expression is measured at the protein level, or protein interaction techniques such as yeast 2-hybrid or mass spectrometry also result in large, highly multivariate datasets, which may be used to generate classifiers and reduced subsets of variables according to the methods disclosed herein. The result of all the classification tasks could be submitted to the same selection in order to define a much reduced set of proteins carrying most of the diagnostic information. One of ordinary skill could then generate a set of monoclonal antibodies for detecting each of the proteins in the reduced subset.

The present invention provides a method for reducing a large complex dataset to a more manageable reduced subset of the most responsive, high impact variables. In many low-throughput diagnostic applications, this reduction is critical to providing a useful analytical device. In some embodiments, this data reduction method may be combined with other information regarding the dataset to develop useful diagnostic devices. For example, a large chemogenomic dataset may be reduced to a subset that is 10% (or less) of the size of the full dataset. This 10% of the high impact, information rich genes may then be further screened or classified to identify those genes whose product is a secreted protein. Secreted proteins in a reduced subset may be identified based on known annotation information regarding the genes in the subset. Because the secreted proteins are identified in the subset of highly responsive genes they are likely to be most useful in protein based diagnostic assays. For example, a monoclonal antibody-based blood serum assay may be prepared based on the subset of genes that produce secreted proteins. Hence, the present invention may be used to generate improved protein-based diagnostic assays from DNA array information.

The general method of the invention as described above is exemplified below. The examples are offered as illustrative embodiments and are not intended to limit the invention.

### EXAMPLES

### Example 1

### Construction of a Multivariate Chemogenomic Dataset (DrugMatrix™)

This example illustrates the construction of a large multivariate chemogenomic dataset based on DNA microarray analysis of rat tissues from over 580 different in vivo compound treatments. This dataset was used to generate signatures comprising genes and weights which subsequently were reduced to yield a subsets of highly responsive genes that may be incorporated into high throughput diagnostic devices as described in Examples 2-7.

The detailed description of the construction of this chemogenomic dataset is described in Examples 1 and 2 of Published U.S. Pat. Appl. No. 2005/0060102 A1, published Mar. 17, 2005, which is hereby incorporated by reference for all purposes. Briefly, in vivo short-term repeat dose rat studies were conducted on over 580 test compounds, including marketed and withdrawn drugs, environmental and industrial toxicants, and standard biochemical reagents. Rats (three per group) were dosed daily at either a low or high dose. The low dose was an efficacious dose estimated from the literature and the high dose was an empirically-determined maximum tolerated dose, defined as the dose that causes a 50% decrease in body weight gain relative to controls during the course of the 5 day range finding study. Animals were necropsied on days 0.25, 1, 3, and 5 or 7. Up to 13 tissues (e.g., liver, kidney, heart, bone marrow, blood, spleen, brain, intestine, glandular and nonglandular stomach, lung, muscle, and gonads) were collected for histopathological evaluation and microarray expression profiling on the Amersham CodeLink™ RU1 platform. In addition, a clinical pathology panel consisting of 37 clinical chemistry and hematology parameters was generated from blood samples collected on days 3 and 5.

In order to assure that all of the dataset is of high quality a number of quality metrics and tests are employed. Failure on any test results in rejection of the array and exclusion from the data set. The first tests measure global array parameters: (1) average normalized signal to background, (2) median signal to threshold, (3) fraction of elements with below background signals, and (4) number of empty spots. The second battery of tests examines the array visually for unevenness and agreement of the signals to a tissue specific reference standard formed from a number of historical untreated animal control arrays (correlation coefficient >0.8). Arrays that pass all of these checks are further assessed using principle component analysis versus a dataset containing seven different tissue types; arrays not closely clustering with their appropriate tissue cloud are discarded.

Data collected from the scanner is processed by the Dewarping/Detrending™ normalization technique, which uses a non-linear centralization normalization procedure (see, Zien, A., T. Aigner, R. Zimmer, and T. Lengauer. 2001. Centralization: A new method for the normalization of gene expression data. Bioinformatics) adapted specifically for the CodeLink microarray platform. The procedure utilizes detrending and dewarping algorithms to adjust for non-biological trends and non-linear patterns in signal response, leading to significant improvements in array data quality.

Log₁₀-ratios are computed for each gene as the difference of the averaged logs of the experimental signals from (usually) three drug-treated animals and the averaged logs of the control signals from (usually) 20 mock vehicle-treated animals. To assign a significance level to each gene expression change, the standard error for the measured change between the experiments and controls is computed. An empirical Bayesian estimate of standard deviation for each measurement is used in calculating the standard error, which is a weighted average of the measurement standard deviation for each experimental condition and a global estimate of measurement standard deviation for each gene determined over thousands of arrays (Carlin, B. P. and T. A. Louis. 2000. “Bayes and empirical Bayes methods for data analysis,” Chapman & Hall/CRC, Boca Raton; Gelman, A. 1995. “Bayesian data analysis, ”Chapman & Hall/CRC, Boca Raton). The standard error is used in a t-test to compute a p-value for the significance of each gene expression change. The coefficient of variation (CV) is defined as the ratio of the standard error to the average Log₁₀-ratio, as defined above.

### Example 2

### Generation of 116 Non-Redundant Signatures

This example illustrates the analysis of the chemogenomic dataset described in Example 1 to yield a set of 116 non-redundant signatures for answering chemogenomic classification questions in liver tissue.

A. Dataset Analysis using a Comprehensive Set of Classification Questions

The subset of 311 compound treatments measured in rat liver tissue from the chemogenomic dataset described in Example 1 was queried with thousands of initial classification questions in a systematic fashion. The classification questions were of four general types:
1. Compound structure-activity relationship (SAR) class versus those not in the SAR class.
2. Compounds exhibiting a specific pharmacological activity (e.g. enzyme inhibition or receptor binding) versus those that do not.
3. Compounds exhibiting a specific clinical chemistry property (e.g. increased metabolite blood serum level) versus those that do not.
4. Compounds resulting in a specific histopathology versus those that do not.

Specifically, an attempt was made to generate a signature for every known compound class, pharmacology, clinical chemistry or histopathology associated with the compounds used to construct the dataset. As described below, the SPLP algorithm was used to generate linear classifiers (i.e. signatures) for each classification question. A threshold performance of logodds ratio>4.00, % TP>=20% and % TN>=97% was required to accept a classifier so generated as a valid signature for answering the classification question.

B. Signature Derivation

To derive each signature, a three-step process of data reduction, signature generation and cross-validation was used. A total of 8565 probes from the total of 10,000 on the Amersham CodeLink™ RU1 microarray were pre-selected based on having less than 5% missing values (e.g. invalid measurement or below signal threshold) in either the positive or negative class of the training set. Pre-selection of these genes increases the quality of the starting dataset but is not necessary in order to generate valid signatures according to the methods disclosed herein. The 8565 genes in the pre-selected set are disclosed in Table 7, which is disclosed in the ASCII formatted file named “Table_{—}7.txt” included on the accompanying CD, which is hereby incorporated by reference herein.

The robust linear programming SVM algorithm SPLP was used to attempt to generate a linear classifier capable of classifying the expression data from the chemogenomic dataset for those compound treatments in the positive class (i.e., +1 labeled data) from the data in the negative class (−1 labeled). This signature generation method is described in PCT Publication No. WO 2004/037200, which is hereby incorporated by reference herein in its entirety. Briefly, the SVM algorithm finds an optimal linear combination of variables (i.e. gene expression measurements) that best separate the two classes of experiments in m dimensional space, where m is equal to 8565. The general form of this linear-discriminant based classifier is defined by n variables: x₁, x₂, . . . xₙ and n associated constants (i.e. weights): a₁, a₂, . . . aₙ, such that:

$$S = {\sum }_{i}^{n} {a}_{i} {x}_{i} - b$$

where S is the scalar product and b is the bias term. Evaluation of S for a test experiment across the n genes in the signature determines what side of the hyperplane in m dimensional space the test experiment lies, and thus the result of the classification. Experiments with scalar products greater than 0 were considered positive for the specific classification question.

C Signature Validation

Cross-validation provides a reasonable approximation of the estimated performance on independent test samples. As described in PCT Publication No. WO 2004/037200, each signature was trained and validated using a 60/40 split sample cross validation procedure. Within each partition of the data set, 60% of the positives and 40% of the negatives were randomly selected and used as a training set to derive a unique signature, which was subsequently used to classify the remaining test cases of known label. This process was repeated 20 times, and the overall performance of the signature was measured as the percent true positive and true negative rate averaged over the 20 partitions of the data set. Splitting the dataset by other fractions or by leave-one-out cross validation gave similar performance estimates.

D. Results: 439 Valid Signatures

A total of 439 valid signatures were generated from the complete set of rat liver tissue data. Each signature comprises a summation of the product of expression logratio values for and associated weighting factors for a set of specific genes. Table 2 (which is disclosed in the ASCII formatted file named “Table_{—}2.txt” included on the accompanying CD, which is hereby incorporated by reference herein) lists information characterizing the 439 classification questions (i.e. pharmacological, toxicological, histopathological states or compound structural classes) that resulted in valid signatures.

As shown in Table 2 (included as the ASCII formatted file named “Table_{—}2.txt” included on the accompanying CD), the “signature description” column lists an abbreviated name or description for the particular classification. “Tissue” indicates the tissue from which the signature was derived. Generally, the gene signature works best for classifying gene expression data from tissue samples from which it was derived. In the present example, all 439 signatures generated are valid in liver tissue. The “Universe Description” is a description of the samples that will be classified by the signature. The chemogenomic dataset described in Example 1 contains information from several tissue types at multiple doses and multiple time points. In order to derive gene signatures it is often useful to restrict classification to only parts of the dataset. So for example, it often is useful to restrict classification to a signature tissue. Other common restrictions are to specific time points, for example day 3 or day 5 time points. The “Universe Description” contains phrases like “Tissue=Liver and Timepoint>=3” which, translates into a restriction that the signature will be derived from compound treatments measured by gene expression analysis of liver tissue on days 3, 5 or 7 (or later if available). Other phrases might say, “Not (Activity_Class_Union=***BLANK***)” which translates into a restriction that any treatment for which the compound has not been annotated with an “Activity_Class_Union” be excluded from the Universe definition. “Class +1 Description” lists descriptions of the definition of the compound treatments in the chemogenomic database that were labeled in the positive group for deriving the signature. “Class −1 description” is the description of the compound treatments that were labeled as not in the class for deriving the signature. “Class 0 description” are the compound treatments that were not used to derive the gene signature. The 0 label is used to exclude compounds for which the +1 or −1 label is ambiguous. For example, in the case of a literature pharmacology signature, there are cases where the compound is neither an agonist or an antagonist but rather a partial agonist. In this case, the safe assumption is to derive a gene signature without including the gene expression data for this compound treatment. Then the gene signature may be used to classify the ambiguous compound after it has been derived. “LOR” refers to the average logodds ratio which is a measure of the performance of each signature.

As listed in Table 2 (included as the ASCII formatted file named “Table_{—}2.txt” included on the accompanying CD), there are several different types of class descriptions used to characterize the classification questions. “Structure Activity Class” (SAC) is a description of both the chemical structure and the pharmacological activity of the compound. Thus, for example, estrogen receptor agonists form one group. Another example: bacterial DNA gyrase inhibitor, 8-fluoro-fluoroquinolone and 8-alkoxy-fluoroquinolone antibiotics each form separate SAC classes even though both share the same pharmacological target, DNA gyrase. “Activity_Class_Union” (also referred to as “Union Class”) is a higher level description of several SAC classes. For example, the DNA gyrase Union Class would include both 8-fluoro-fluoroquinolone and 8-alkoxy-fluoroquinolone antibiotics.

Compound activities are also referred to in the class descriptions listed in Table 2 (included as the ASCII formatted file named “Table_{—}2.txt” included on the accompanying CD). The exact assay referred to in each activity measurement is encoded as “IC50-XXXXX|Assay name,” where xxxxx is the catalog number for the assay in the MDS-Pharma Services on-line catalog found at URL “discovery.mdsps.com/catalog”. Thus, for example, “IC50-219501|Dopamine D1” indicates the Dopamine D1 assay with the MDS catalog number 21950. All compound activities are reported as −log(IC50), where the IC50 is reported in μM. Therefore, “>=0.000000000001” indicates that the value should be greater than zero and thus greater than 1 μm (i.e. since log(1 μM)=0). Furthermore, the testing protocols used in constructing the database of Example 1 did not determine IC50 values greater than about 35 μM. All cases where the IC50 was estimated to be greater than 35 μm was recorded in the database as “−3” (i.e. the IC50 was considered to be 1 mM and thus, −log(1000 μM)=−3). This number implies that the compound does not bind to the site under investigation.

E. Producing a Set of 116 Maximally Divergent, Non Redundant Signatures

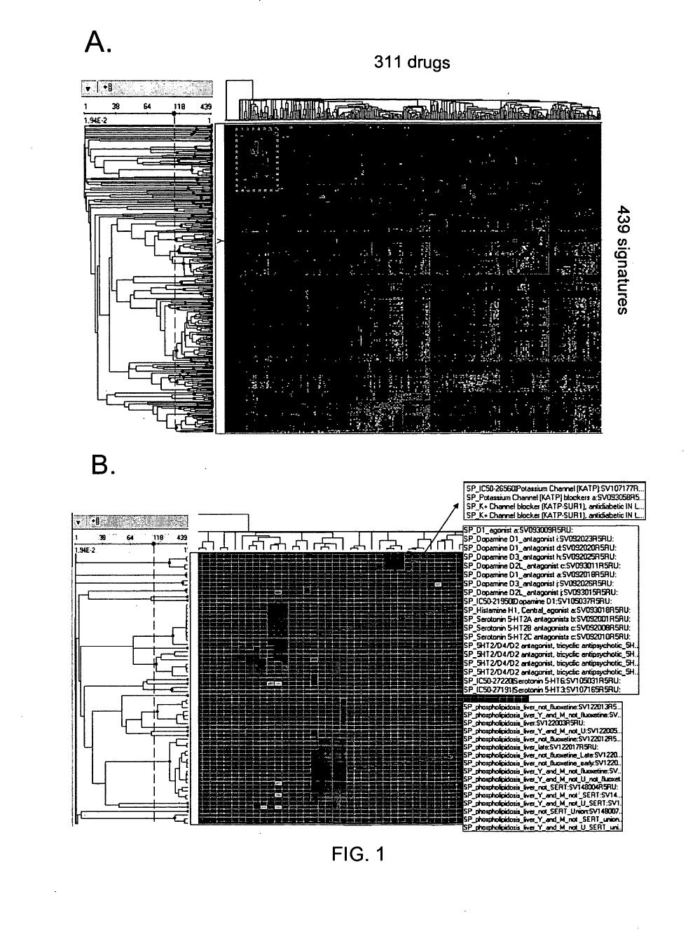

The set of 439 gene signatures listed in Table 2 (included as the ASCII formatted file named “Table_{—}2.txt” included on the accompanying CD) was further reduced to a smaller set of 116 non-redundant signatures. FIG. 1A depicts a plot of each of 311 treatments (each treatment including two dosage levels at four time points) of rats (x-axis) versus the scalar product (see, below) for that treatment's effect on the RNA expression profile of the genes in each of 439 derived signatures (y-axis). Each signature was represented by its maximum scalar product under any condition for a given drug treatment. Each signature represents a “classification question” for which a valid SPLP classification signature (i.e. minimal performance: LOR>4.0), could be derived, based on a liver gene expression database comprising treatments of rats with 311 compounds at a maximum tolerated dose or a fully effective dose, and measurements at 0.25 days, 1 day, 3 days and 5 days of once daily dosing. Only positive values were used for clustering; negative values have been reset to 0. The clustering method was UPGMA and the Pearson's correlation coefficient was used as a distance metric.

The vertical dashed line through the cluster “trees” along the y-axis indicates the position corresponding to correlation ˜0.7. Slicing the trees of signatures in that position defined 116 clusters. A single signature (the one having the highest test logodds ratio) was chosen from each cluster as representative of that signature group and of a specific biological event distinguishable from other biological events caused by compound treatments.

FIG. 1B illustrates how one of the 116 non-redundant signatures is representative of several signatures. FIG. 1B depict a small subset of clustered signatures and treatments in the upper left corner of FIG. 1A. The uppermost cluster depicted in FIG. 1B is composed of various signatures for potassium channel blockers. This cluster, as well as the bottom cluster of phospholipodosis signatures is represented by a single signature in the list of 116 non redundant signatures because the 0.7 correlation threshold defines a single group (see dashed line through the cluster “trees” along the y-axis). The middle group composed mostly of signatures serotonin, dopamine and histamine receptor interacting compounds is composed of three sub-clusters.

The 116 classification questions that generated the non-redundant signatures are listed in Table 3. The 116 non-redundant signatures utilize only 3421 of the 8565 genes present on the DNA microarrays used to generate the original chemogenomic dataset. This reduction from 439 to 116 signatures (including only 3421 different genes) suggests that a reduced subset of less than half of the genes in the original dataset may be utilized to answer all of the classification questions within the scope of the original queries.

TABLE 3
116 Non-Redundant Gene Signatures in LIVER

| Cluster | Universe | Class 1 | Class −1 | Class 0 | Avg. |
|---|---|---|---|---|---|
| No. | Description | Description | Description | Description | LOR |
| 1 | (Tissue = LIVER) | (STRUCTURE_{—} | All else | (Zero_Class = | 4.24 |
| | Not (STRUCTURE_{—} | ACTIVITY = | | ***Blank***) Or | |
| | ACTIVITY = | Estrogen receptor | | (Zero_Class = Y) | |
| | ***Blank***) | agonist, | | | |
| | | environmental | | | |
| | | toxicant | | | |
| 2 | (Tissue = LIVER And | (ACTIVITY_CLASS_{—} | All else | (Zero_Class = | 5.41 |
| | TimePoint >= 3) | UNION = | | ***Blank***) Or | |
| | Not (ACTIVITY_CLASS_{—} | Bacterial folate | | (Zero_Class = Y) | |
| | UNION = | synthesis inhibitor, | | | |
| | ***Blank***) | dihydropteroate | | | |
| | | synthase inhibitor_{—} | | | |
| | | Bacterial folate | | | |
| | | synthesis inhibitor, | | | |
| | | dihydropteroate | | | |
| | | synthase inhibitor, | | | |
| | | isoxazol- | | | |
| | | sulfonamide_Bacterial | | | |
| | | folate synthesis | | | |
| | | inhibitor, | | | |
| | | dihydropteroate | | | |
| | | synthase inhibitor, | | | |
| | | pyrimidin-sulfonamide | | | |
| 3 | (Tissue = LIVER And | (STRUCTURE_{—} | All else | (Zero_Class = | 5.96 |
| | TimePoint >= 3) | ACTIVITY = | | ***Blank***) Or | |
| | Not (STRUCTURE_{—} | Toxicant, heavy metal | | (Zero_Class = Y) | |
| | ACTIVITY = | | | | |
| | ***Blank***) | | | | |
| 4 | (Tissue = LIVER And | (ACTIVITY_CLASS_{—} | All else | (Zero_Class = | 4.41 |
| | HighOrLowDose = HI) | UNION = | | ***Blank***) Or | |
| | Not (ACTIVITY_CLASS_{—} | Voltage gated Na+ | | (Zero_Class = Y) | |
| | UNION = | channel blocker, | | | |
| | ***Blank***) | alpha-amino-acid | | | |
| | | arylamide_Voltage | | | |
| | | gated Na+ | | | |
| | | channel blocker, | | | |
| | | anticonvulsant_Voltage | | | |
| | | gated Na+ channel | | | |
| | | blocker, lipophylic | | | |
| | | amine_Voltage | | | |
| | | gated Na+ channel | | | |
| | | blocker, | | | |
| | | p-aminobenzoate | | | |
| 5 | (Tissue = LIVER) | (ACTIVITY_CLASS_{—} | All else | (Zero_Class = | 6.06 |
| | Not (ACTIVITY_CLASS_{—} | UNION = | | ***Blank***) Or | |
| | UNION = | H2O2 radical | | (Zero_Class = Y) | |
| | ***Blank***) | scavenger | | | |
| 6 | (Tissue = LIVER And | (STRUCTURE_{—} | All else | (Zero_Class = | 5.01 |
| | HighOrLowDose = | ACTIVITY = | | ***Blank***) Or | |
| | HI) Not (STRUCTURE_{—} | NSAID, COX-3, | | (Zero_Class = Y) | |
| | ACTIVITY = | acetaminophen like | | | |
| | ***Blank***) | | | | |
| 7 | (Tissue = LIVER) | (IC50-26560\| | (IC50-26560\| | All else | 5.92 |
| | Not (IC50-26560\| | Potassium Channel | Potassium Channel | | |
| | Potassium Channel | [KATP] >= −1) | [KATP] = −3) | | |
| | [KATP] = | Not (MDS_Specific_{—} | Or (MDS_Specific_{—} | | |
| | ***Blank***) | Groupings_B = | Groupings_B = | | |
| | | K+_channel_opener) | K+_channel_opener) | | |
| 8 | (Tissue = LIVER And | (STRUCTURE_{—} | All else | (Zero_Class = | 6.04 |
| | TimePoint >= 3) | ACTIVITY = | | ***Blank***) Or | |
| | Not (STRUCTURE_{—} | Bacterial ribosomal | | (Zero_Class = Y) | |
| | ACTIVITY = | (30S) function | | | |
| | ***Blank***) | inhibitor, | | | |
| | | tetracycline | | | |
| 9 | (Tissue = LIVER) | (STRUCTURE_{—} | All else | (Zero_Class = | 6.79 |
| | Not (STRUCTURE_{—} | ACTIVITY = | | ***Blank***) Or | |
| | ACTIVITY = | GABA-A agonist, | | (Zero_Class = Y) | |
| | ***Blank***) | non-NMDA-glutamate | | | |
| | | antagonist, Voltage- | | | |
| | | dependent Ca++ | | | |
| | | channel blocker, | | | |
| | | barbiturate | | | |
| 10 | (Tissue = LIVER) | (ACTIVITY_CLASS_{—} | All else | (Zero_Class = | 6.81 |
| | Not (ACTIVITY_CLASS_{—} | UNION = | | ***Blank***) Or | |
| | UNION = | Histamine receptor | | (Zero_Class = Y) | |
| | ***Blank***) | (H1) antagonist_{—} | | | |
| | | Histamine receptor | | | |
| | | (H1) antagonist, | | | |
| | | adenosine receptor | | | |
| | | antagonist_Histamine | | | |
| | | receptor (H1) | | | |
| | | antagonist, Ca++ | | | |
| | | channel (L-Type) | | | |
| | | blocker_Histamine | | | |
| | | receptor (H1) | | | |
| | | antagonist, | | | |
| | | diphenylamine_Histamine | | | |
| | | receptor (H1) antagonist, | | | |
| | | hepatocarcinogen_{—} | | | |
| | | Histamine receptor (H1) | | | |
| | | antagonist, tricyclic_{—} | | | |
| | | Histamine receptor | | | |
| | | (H2) antagonist | | | |
| 11 | (Tissue = LIVER) | (ACTIVITY_CLASS_{—} | All else | (Zero_Class = | 4.95 |
| | Not (ACTIVITY_{—} | UNION = | | ***Blank***) Or | |
| | CLASS_UNION = | Ca++ channel | | (Zero_Class = Y) | |
| | ***Blank***) | (L-Type) blocker_{—} | | | |
| | | Ca++ channel | | | |
| | | (L-Type) blocker, 1,4-DHP_{—} | | | |
| | | Ca++ channel | | | |
| | | (T-Type) blocker_{—} | | | |
| | | Ca++ channel | | | |
| | | blocker, antiparasitics | | | |
| 12 | (Tissue = LIVER) | >=0.0000000000001 | −3 | All else | 4.25 |
| 13 | (Tissue = LIVER And | (ACTIVITY_CLASS_{—} | All else | (Zero_Class = | 7.43 |
| | TimePoint >= 3) | UNION = | | ***Blank***) Or | |
| | Not (ACTIVITY_{—} | 5HT2/D4/D2 antagonist, | | (Zero_Class = Y) | |
| | CLASS_UNION = | tricyclic antipsychotic_{—} | | | |
| | ***Blank***) | 5HT2/D4/D2 antagonist, | | | |
| | | tricyclic antipsychotic_{—} | | | |
| | | 5HT2/H1 antagonist, | | | |
| | | tricyclic_5HT3 | | | |
| | | antagonist | | | |
| 14 | (Tissue = LIVER) | >−1 Not ***Blank*** | −3 | All else | 4.75 |
| 15 | (Tissue = LIVER) | (IC50-21950\| | (IC50-21950\| | All else | 4.95 |
| | Not (IC50-21950\| | Dopamine D1 >= 0) | Dopamine | | |
| | Dopamine D1 = | Not (MDS_Specific_{—} | D1 = −3) | | |
| | ***Blank***) | Groupings_A = | Or (MDS_Specific_{—} | | |
| | | D_agonist) | Groupings_A = | | |
| | | | D_agonist) | | |
| 16 | (Tissue = LIVER) | (IC50-21980\| | (IC50-21980\| | All else | 4.79 |
| | Not (IC50-21980\| | Dopamine D3 >= −1 | Dopamine | | |
| | Dopamine D3 = | And New_Activity_{—} | D3 = − 3) | | |
| | ***Blank***) | Class = | Or (MDS_Specific_{—} | | |
| | | Dopamine receptor | Groupings_A = | | |
| | | antagonist (D), | D_agonist) | | |
| | | phenothiazine) | | | |
| | | Not (MDS_Specific_{—} | | | |
| | | Groupings_A = | | | |
| | | D_agonist) | | | |
| 17 | (Tissue = LIVER) | (IC50-27170\|Serotonin | (IC50-27170\| | All else | 4.93 |
| | Not (IC50-27170\| | 5-HT2B => −1 | Serotonin | | |
| | Serotonin 5-HT2B = | And New_Activity_{—} | 5-HT2B = −3) | | |
| | ***Blank***) | Class_Unions = | Or (MDS_Specific_{—} | | |
| | | Monoamine Re-uptake | Groupings_A = | | |
| | | (DAT) inhibitor_{—} | 5HT_agonist) | | |
| | | union_Monoamine Re- | | | |
| | | uptake (NET/SERT) | | | |
| | | inhibitor, tricyclic_{—} | | | |
| | | union_Monoamine Re- | | | |
| | | uptake (SERT) inhibitor, | | | |
| | | heterogeneous structures) | | | |
| | | Not (MDS_Specific_{—} | | | |
| | | Groupings_A = | | | |
| | | 5HT_agonist) | | | |
| 18 | (Tissue = LIVER) | (IC50-27165\|Serotonin | (IC50-27165\| | All else | 6.42 |
| | Not (IC50-27165\| | 5-HT2A >= −1 | Serotonin | | |
| | Serotonin 5-HT2A = | And New_Activity_{—} | 5-HT2A = −3) | | |
| | ***Blank***) | Class_Unions = | Or (MDS_Specific_{—} | | |
| | | Monoamine Re-uptake | Groupings_A = | | |
| | | (DAT) inhibitor_union_{—} | 5HT_agonist) | | |
| | | Monoamine Re-uptake | | | |
| | | (NET/SERT) inhibitor, | | | |
| | | tricyclic_union_{—} | | | |
| | | Monoamine Re-uptake | | | |
| | | (SERT) inhibitor, | | | |
| | | heterogeneous structures) | | | |
| | | Not (MDS_Specific_{—} | | | |
| | | Groupings_A = | | | |
| | | 5HT_agonist) | | | |
| 19 | (Tissue = LIVER And | (STRUCTURE_{—} | All else | (Zero_Class = | 5.86 |
| | HighOrLowDose = HI) | ACTIVITY = | | ***Blank***) Or | |
| | Not (STRUCTURE_{—} | Monoamine Re-uptake | | (Zero_Class = Y) | |
| | ACTIVITY = | (SERT) inhibitor, | | | |
| | ***Blank***) | heterogeneous structures | | | |
| 20 | (Tissue = LIVER) | >=0.0000000000001 | −3 | All else | 6.49 |
| 21 | (Tissue = LIVER And | (STRUCTURE_{—} | All else | (Zero_Class = | 10.92 |
| | TimePoint >= 3) | ACTIVITY = | | ***Blank***) Or | |
| | Not (STRUCTURE_{—} | Estrogen receptor | | (Zero_Class = Y) | |
| | ACTIVITY = | antagonist/agonist, | | | |
| | ***Blank***) | tissue specific | | | |
| 22 | (Tissue = LIVER) | (ACTIVITY_CLASS_{—} | All else | (Zero_Class = | 5.29 |
| | Not (ACTIVITY_CLASS_{—} | UNION = | | ***Blank***) Or | |
| | UNION = | Estrogen antagonist, | | (Zero_Class = Y) | |
| | ***Blank***) | aromatase inhibitor_{—} | | | |
| | | Estrogen receptor | | | |
| | | antagonist/agonist, | | | |
| | | tissue specific | | | |
| 23 | (Tissue = LIVER) | >=0.0000000000001 | −3 | All else | 4.67 |
| 24 | (Tissue = LIVER) | >=0.0000000000001 | −3 | All else | 4.58 |
| 25 | (Tissue = LIVER) | >=0.0000000000001 | −3 | All else | 4.34 |
| 26 | (Tissue = LIVER) | (PHOSPHOLIPIDOSIS = Y) | All else | (Drug = | 5.52 |
| | | Not (Drug = | | FLUOXETINE) | |
| | | FLUOXETINE) | | | |
| 27 | (Tissue = LIVER) | (STRUCTURE_{—} | All else | (Zero_Class = | 4.17 |
| | Not (STRUCTURE_{—} | ACTIVITY = | | ***Blank***) Or | |
| | ACTIVITY = | Dopamine receptor | | (Zero_Class = Y) | |
| | ***Blank***) | antagonist (D), | | | |
| | | phenothiazine | | | |
| 28 | (Tissue = LIVER) | (IC50-25270\| | (IC50-25270\| | All else | 4.18 |
| | Not (IC50-25270\| | Muscarinic M2 >= 0) | Muscarinic | | |
| | Muscarinic M2 = | Not (New Activity_Class_{—} | M2 = −3) Or | | |
| | ***Blank***) | Unions = | (New_Activity_Class_{—} | | |
| | | Muscarinic acetylcoline | Unions = | | |
| | | receptor (M) agonist) | Muscarinic acetylcoline | | |
| | | | receptor (M) agonist) | | |
| 29 | (Tissue = LIVER And | (STRUCTURE_{—} | All else | (Zero_Class = | 4.33 |
| | HighOrLowDose = HI) | ACTIVITY = | | ***Blank***) Or | |
| | Not (STRUCTURE_{—} | Bacterial ribosomal | | (Zero_Class = Y) | |
| | ACTIVITY = | (50S) function | | | |
| | ***Blank***) | inhibitor, macrolide | | | |
| 30 | (Tissue = LIVER And | (ACTIVITY_CLASS_{—} | All else | (Zero_Class = | 6.18 |
| | HighOrLowDose = HI | UNION = | | ***Blank***) Or | |
| | And TimePoint >= 3) | Bacterial DNA gyrase | | (Zero_Class = Y) | |
| | Not (ACTIVITY_CLASS_{—} | inhibitor, 8-alkoxy- | | | |
| | UNION = | fluoroquinolone_{—} | | | |
| | ***Blank***) | Bacterial DNA gyrase | | | |
| | | inhibitor, 8-fluoro- | | | |
| | | fluoroquinolone_{—} | | | |
| | | Bacterial DNA gyrase | | | |
| | | inhibitor, | | | |
| | | 8-N-fluoroquinolone_{—} | | | |
| | | Bacterial DNA | | | |
| | | gyrase inhibitor, | | | |
| | | fluoroquinolone | | | |
| 31 | (Tissue = LIVER And | (ACTIVITY_CLASS_{—} | All else | (Zero_Class = | 6.40 |
| | HighOrLowDose = HI) | UNION = | | ***Blank***) Or | |
| | Not (ACTIVITY_CLASS_{—} | Thyroperoxidase | | (Zero_Class = Y) | |
| | UNION = | inhibitor | | | |
| | ***Blank***) | | | | |
| 32 | (Tissue = LIVER) | (IC50-22601\| | (IC50-22601\| | All else | 5.53 |
| | Not (IC50-22601\| | Estrogen | Estrogen | | |
| | Estrogen ERalpha = | ERalpha >= 0) | ERalpha = −3) | | |
| | ***Blank***) | Not (MDS_Specific_{—} | Or (MDS_Specific_{—} | | |
| | | Groupings_A = | Groupings_A = | | |
| | | Estrogen_agonist) | Estrogen_agonist) | | |
| 33 | (Tissue = LIVER) | (TISSUE_{—} | All else | (Zero_Class = | 6.54 |
| | Not (TISSUE_{—} | TOXICITY = | | ***Blank***) Or | |
| | TOXICITY = | Hepatocellular | | (Zero_Class = Y) | |
| | ***Blank***) | Carcinoma) | | | |
| 34 | (Tissue = LIVER) | (IC50-28501\| | (IC50-28501\| | All else | 6.43 |
| | Not (IC50-28501\| | Testosterone >= 0 | Testosterone = −3) | | |
| | Testosterone = | And MDS_Specific_{—} | Or (MDS_Specific_{—} | | |
| | ***Blank***) | Groupings_A = | Groupings_A = | | |
| | | Androgen_agonist) | Androgen_antagonist) | | |
| | | Not (MDS_Specific_{—} | | | |
| | | Groupings_A = | | | |
| | | Androgen_antagonist) | | | |
| 35 | (Tissue = LIVER And | 95th % | 0-75th % | rest | 4.07 |
| | TimePoint >= 5 And | | | | |
| | ClinicalChemInfo = Y) | | | | |
| 36 | (Tissue = LIVER And | (ACTIVITY_CLASS_{—} | All else | (Zero_Class = | 7.75 |
| | HighOrLowDose = HI) | UNION = | | ***Blank***) Or | |
| | Not (ACTIVITY_CLASS_{—} | H+/K+-ATPase | | (Zero_Class = Y) | |
| | UNION = | inhibitor | | | |
| | ***Blank***) | | | | |
| 37 | (Tissue = LIVER And | (STRUCTURE_{—} | All else | (Zero_Class = | 5.02 |
| | TimePoint >= 3) | ACTIVITY = | | ***Blank***) Or | |
| | Not (STRUCTURE_{—} | Tubulin binder, | | (Zero_Class = Y) | |
| | ACTIVITY = | benzimidazole | | | |
| | ***Blank***) | | | | |
| 38 | (Tissue = LIVER) | (STRUCTURE_{—} | All else | (Zero_Class = | 5.05 |
| | Not (STRUCTURE_{—} | ACTIVITY = | | ***Blank***) Or | |
| | ACTIVITY = | DNA damaging, free | | (Zero_Class = Y) | |
| | ***Blank***) | oxygen radical | | | |
| | | generator | | | |
| 39 | (Tissue = LIVER And | LIVER carcinogens | ALL ELSE | BLIND, AVENTIS | 8.19 |
| | TimePoint >= 3 | and genotoxic, | | | |
| | but <= 5) | d3 and d5 | | | |
| 40 | (Tissue = LIVER And | (STRUCTURE_{—} | All else | (Zero_Class = | 5.01 |
| | HighOrLowDose = HI) | ACTIVITY = | | ***Blank***) Or | |
| | Not (STRUCTURE_{—} | Estrogen antagonist, | | (Zero_Class = Y) | |
| | ACTIVITY = | aromatase inhibitor | | | |
| | ***Blank***) | | | | |
| 41 | (TISSUE = LIVER And | Day5_LIVER- | Day5_LIVER- | all else | 5.10 |
| | TimePoint >= 0.25 | CENTROLOBULAR | CENTROLOBULAR | | |
| | but <= 1 And | HYDROPIC CHANGE | HYDROPIC CHANGE | | |
| | Day5_LIVER- | SEVERITY SCORE > 2 in | SEVERITY SCORE = | | |
| | CENTROLOBULAR | at least 1 animal(s) | 0 in all animals | | |
| | HYDROPIC CHANGE = Y) | | | | |
| 42 | (Tissue = LIVER And | (STRUCTURE_{—} | All else | (Zero_Class = | 4.85 |
| | HighOrLowDose = HI | ACTIVITY = | | ***Blank***) Or | |
| | And TimePoint >= 3) | GABA-A agonist, | | (Zero_Class = Y) | |
| | Not (STRUCTURE_{—} | benzodiazepin, | | | |
| | ACTIVITY = | long acting | | | |
| | ***Blank***) | | | | |
| 43 | (Tissue = LIVER) | (IC50-22660\| | All else | (MDS_Specific_{—} | 4.98 |
| | Not (IC50-22660\| | GABAA, | | Groupings_A = | |
| | GABAA, | Benzodiazepine, | | GABA_agonist_{—} | |
| | Benzodiazepine, | Central >= −1 | | channel) Or (New_{—} | |
| | Central = | And MDS_Specific_{—} | | Activity_Class = | |
| | ***Blank***) | Groupings_A = | | GABA-B agonist) | |
| | | GABA_agonist_timed) | | | |
| 44 | (Tissue = LIVER) | (STRUCTURE_{—} | All else | (Zero_Class = | 5.21 |
| | Not (STRUCTURE_{—} | ACTIVITY = | | ***Blank***) Or | |
| | ACTIVITY = | Pro-inflammatory | | (Zero_Class = Y) | |
| | ***Blank***) | stimuli | | | |
| 45 | (Tissue = LIVER) | >−1 | −3 | All else | 4.48 |
| | | Not ***Blank*** | | | |
| 46 | (Tissue = LIVER) | (TISSUE_{—} | All else | (Zero_Class = | 4.96 |
| | Not (TISSUE_{—} | TOXICITY = | | ***Blank***) Or | |
| | TOXICITY = | Embryotoxicity) | | (Zero_Class = Y) | |
| | ***Blank***) | | | | |
| 47 | (Tissue = LIVER) | (TISSUE_{—} | All else | (Zero_Class = | 6.37 |
| | Not (TISSUE_{—} | TOXICITY = | | ***Blank***) Or | |
| | TOXICITY = | Fetal Toxicity) | | (Zero_Class = Y) | |
| | ***Blank***) | | | | |
| 48 | (Tissue = LIVER) | >=0.0000000000001 | −3 | All else | 5.58 |
| 49 | (Tissue = LIVER And | (STRUCTURE_{—} | All else | (Zero_Class = | 5.11 |
| | HighOrLowDose = HI) | ACTIVITY = | | ***Blank***) Or | |
| | Not (STRUCTURE_{—} | NSAID, COX-2/1, | | (Zero_Class = Y) | |
| | ACTIVITY = | coxib like | | | |
| | ***Blank***) | | | | |
| 50 | (Tissue = LIVER) | (STRUCTURE_{—} | All else | (Zero_Class = | 5.77 |
| | Not (STRUCTURE_{—} | ACTIVITY = | | ***Blank***) Or | |
| | ACTIVITY = | PPAR alpha agonist | | (Zero_Class = Y) | |
| | ***Blank***) | | | | |
| 51 | (Tissue = LIVER) | (STRUCTURE_{—} | All else | (Zero_Class = | 7.39 |
| | Not (STRUCTURE_{—} | ACTIVITY = | | ***Blank***) Or | |
| | ACTIVITY = | PPAR alpha agonist, | | (Zero_Class = Y) | |
| | ***Blank***) | fibrate | | | |
| 52 | (Tissue = LIVER) | (ACTIVITY_CLASS_{—} | All else | (Zero_Class = | 9.39 |
| | Not (ACTIVITY_CLASS_{—} | UNION = | | ***Blank***) Or | |
| | UNION = | PPAR alpha agonist_{—} | | (Zero_Class = Y) | |
| | ***Blank***) | PPAR alpha | | | |
| | | agonist, fibrate | | | |
| 53 | (Tissue = LIVER) | >−1 Not ***Blank*** | −3 | All else | 5.30 |
| 54 | (TISSUE = LIVER And | Day5_LIVER- | Day5_LIVER-NECROSIS | all else | 4.99 |
| | TimePoint >= 0.25 | NECROSIS SEVERITY | SEVERITY SCORE = | | |
| | but <= 1 And | SCORE > 0 in at | 0 in all animals | | |
| | Day5_LIVER- | least 2 animal(s) | | | |
| | NECROSIS = Y) | | | | |
| 55 | (Tissue = LIVER And | (STRUCTURE_{—} | All else | (Zero_Class = | 4.57 |
| | TimePoint >= 3) | ACTIVITY = | | ***Blank***) Or | |
| | Not (STRUCTURE_{—} | DNA-alkylator | | (Zero_Class = Y) | |
| | ACTIVITY = | | | | |
| | ***Blank***) | | | | |
| 56 | (Tissue = LIVER) | (STRUCTURE_{—} | All else | (Zero_Class = | 4.40 |
| | Not (STRUCTURE_{—} | ACTIVITY = | | ***Blank***) Or | |
| | ACTIVITY = | Toxicant, free | | (Zero_Class = Y) | |
| | ***Blank***) | oxygen radical | | | |
| | | generator | | | |
| 57 | (Tissue = LIVER) | >−1 Not ***Blank*** | −3 | All else | 5.82 |
| 58 | (Tissue = LIVER And | see KK109, long | ALL ELSE | BLIND, AVENTIS | 4.73 |
| | TimePoint >= 3 | term benzodiazepines | | | |
| | but <= 5) | nad phenobarbital | | | |
| | | and estrogens | | | |
| 59 | (Tissue = LIVER And | (ACTIVITY_CLASS_{—} | All else | (Zero_Class = | 5.61 |
| | HighOrLowDose = HI) | UNION = | | ***Blank***) Or | |
| | Not (ACTIVITY_CLASS_{—} | Progesterone receptor | | (Zero_Class = Y) | |
| | UNION = | agonist | | | |
| | ***Blank***) | | | | |
| 60 | (Tissue = LIVER) | >=0.0000000000001 | −3 | All else | 5.03 |
| 61 | (TISSUE = LIVER And | LIPASE <= 5th | LIPASE <= | all else | 5.08 |
| | TimePoint >= 5 | percentile | 65th percentile And | | |
| | but <= 7 And | | LIPASE >= | | |
| | LIPASE = Y) | | 35th percentile | | |
| 62 | (TISSUE = LIVER And | Day 5_CARBON | Day5_CARBON | all else | 4.00 |
| | TimePoint = 0.25 | DIOXIDE <= 5th | DIOXIDE >= | | |
| | And Day5_CARBON | percentile | 35th percentile | | |
| | DIOXIDE = Y) | | | | |
| 63 | (TISSUE = LIVER And | Day5_LIPASE <= 5th | Day5_LIPASE <= | all else | 7.60 |
| | TimePoint = 0.25 | percentile | 65th percentile And | | |
| | And Day5_LIPASE = Y) | | Day5_LIPASE >= | | |
| | | | 35th percentile | | |
| 64 | (Tissue = LIVER) | (TISSUE_{—} | All else | (Zero_Class = | 6.49 |
| | Not (TISSUE_{—} | TOXICITY = | | ***Blank***) Or | |
| | TOXICITY = | Hepatic Adenoma) | | (Zero_Class = Y) | |
| | ***Blank***) | | | | |
| 65 | (Tissue = LIVER And | (ACTIVITY_CLASS_{—} | All else | (Zero_Class = | 9.11 |
| | TimePoint >= 3) | UNION = | | ***Blank***) Or | |
| | Not (ACTIVITY_CLASS_{—} | Estrogen receptor | | (Zero_Class = Y) | |
| | UNION = | agonist_Estrogen | | | |
| | ***Blank***) | receptor agonist, | | | |
| | | steroidal | | | |
| 66 | (Tissue = LIVER) | (IC50-22601\| | (IC50-22601\| | All else | 4.74 |
| | Not (IC50-22601\| | Estrogen | Estrogen | | |
| | Estrogen ERalpha = | ERalpha >= −1 | ERalpha = −3) | | |
| | ***Blank***) | And MDS_Specific_{—} | Or (MDS_Specific_{—} | | |
| | | Groupings_A = | Groupings_A = | | |
| | | Estrogen_agonist) | Estrogen_antagonist) | | |
| | | Not (MDS_Specific_{—} | | | |
| | | Groupings_A = | | | |
| | | Estrogen_antagonist) | | | |
| 67 | (TISSUE = LIVER And | ALKALINE | ALKALINE | all else | 5.62 |
| | TimePoint >= 5 | PHOSPHATASE >= | PHOSPHATASE <= | | |
| | but <= 7 And | 95th percentile | 65th percentile | | |
| | ALKALINE | | | | |
| | PHOSPHATASE = Y) | | | | |
| 68 | (TISSUE = LIVER And | ALKALINE | ALKALINE | all else | 4.95 |
| | TimePoint = 3 And | PHOSPHATASE >= | PHOSPHATASE <= | | |
| | ALKALINE | 95th percentile | 65th percentile | | |
| | PHOSPHATASE = Y) | | | | |
| 69 | (Tissue = LIVER And | 98th % | 25-75th % | rest | 6.04 |
| | TimePoint >= 5 And | | | | |
| | ClinicalChemInfo = Y) | | | | |
| 70 | (TISSUE = LIVER And | CHOLESTEROL <= | CHOLESTEROL >= | all else | 6.51 |
| | TimePoint >= 3 | 5th percentile | 35th percentile | | |
| | but <= 7 And | | | | |
| | CHOLESTEROL = Y) | | | | |
| 71 | (Tissue = LIVER And | (STRUCTURE_{—} | All else | (Zero_Class = | 6.38 |
| | TimePoint >= 3) | ACTIVITY = | | ***Blank***) Or | |
| | Not (STRUCTURE_{—} | DNA damaging, free | | (Zero_Class = Y) | |
| | ACTIVITY = | oxygen radical | | | |
| | ***Blank***) | generator, nitro- | | | |
| | | sourea | | | |
| 72 | (Tissue = LIVER) | | | | 7.16 |
| 73 | (TISSUE = LIVER And | Day5_LIVER- | Day5_LIVER-APOPTOSIS | all else | 4.43 |
| | TimePoint >= 0.25 | APOPTOSIS | SEVERITY SCORE = | | |
| | but <= 1 And | SEVERITY SCORE > 0 | 0 in all animals | | |
| | Day5_LIVER- | in at least 3 | | | |
| | APOPTOSIS = Y) | animal(s) | | | |
| 74 | (Tissue = LIVER And | 98th percentile; | 0-75th percentile; | other | 4.35 |
| | TimePoint >= 5 And | liver; day 5/7 | liver; day 5/7 | | |
| | ClinicalChemInfo = Y) | | | | |
| 75 | (TISSUE = LIVER And | LIVER-HEPATOCYTE | LIVER-HEPATOCYTE | all else | 5.68 |
| | TimePoint >= 3 | ENLARGEMENT | ENLARGEMENT | | |
| | but <= 7 And | SEVERITY SCORE > 2 | SEVERITY SCORE = | | |
| | LIVER-HEPATOCYTE | in at least 3 | 0 in all animals | | |
| | ENLARGEMENT = Y) | animal(s) | | | |
| 76 | (Tissue = LIVER And | (STRUCTURE_{—} | All else | (Zero_Class = | 7.55 |
| | HighOrLowDose = HI | ACTIVITY = | | ***Blank***) Or | |
| | And TimePoint >= 3) | HMG-CoA reductase | | (Zero_Class = Y) | |
| | Not (STRUCTURE_{—} | inhibitors | | | |
| | ACTIVITY = | | | | |
| | ***Blank***) | | | | |
| 77 | (TISSUE = LIVER And | LIVER-APOPTOSIS | LIVER-APOPTOSIS | all else | 4.79 |
| | TimePoint >= 0.25 | SUM OF SEVERITY | SUM OF SEVERITY | | |
| | but <= 7 And | SCORE > 3 | SCORE = 0 | | |
| | LIVER-APOPTOSIS = Y) | | | | |
| 78 | (TISSUE = LIVER And | LIVER-APOPTOSIS | LIVER-APOPTOSIS | all else | 4.24 |
| | TimePoint >= 5 | SEVERITY SCORE > 1 | SEVERITY SCORE = | | |
| | but <= 7 And | in at least 1 animal(s) | 0 in all animals | | |
| | LIVER-APOPTOSIS = Y) | | | | |
| 79 | (Tissue = LIVER And | 98th % | 25-75th % | rest | 4.37 |
| | TimePoint >= 5 And | | | | |
| | ClinicalChemInfo = Y) | | | | |
| 80 | (Tissue = LIVER And | 98th percentile; | 0-75th percentile; | other | 5.74 |
| | TimePoint >= 5 And | liver; day 5/7 | liver; day 5/7 | | |
| | ClinicalChemInfo = Y) | | | | |
| 81 | (Tissue = LIVER And | All liver REPIDS where | ALL ELSE where ALT or | BLIND, AVENTIS | 7.15 |
| | TimePoint >= 3 | ALT, AP, and | AP or BIL are < 1.5 | | |
| | but <= 5 And | Bilirubin > 1.5 fold | | | |
| | ClinicalChemInfo = Y) | increased | | | |
| 82 | (Tissue = LIVER And | 98th % | 25-75th % | rest | 4.63 |
| | TimePoint >= 5 And | | | | |
| | ClinicalChemInfo = Y) | | | | |
| 83 | (Tissue = LIVER And | ALL REPIDS where weight | ALL REPIDS where weight | other tissues and | 4.55 |
| | Body_Weight_Info = Y) | change is < 25% loss | change is > 10% loss | remaining liver REPIDS | |
| 84 | (Tissue = LIVER And | 98th % | 25-75th % | rest | 6.96 |
| | TimePoint >= 5 And | | | | |
| | ClinicalChemInfo = Y) | | | | |
| 85 | (Tissue = LIVER And | 98th percentile; | 25-75th percentile; | other | 5.44 |
| | TimePoint >= 5 And | liver; day 5/7 | liver; day 5/7 | | |
| | ClinicalChemInfo = Y) | | | | |
| 86 | (TISSUE = LIVER And | ASPARTATE | ASPARTATE | all else | 7.29 |
| | TimePoint >= 0.25 | AMINOTRANSFERASE >= 95th | AMINOTRANSFERASE | | |
| | but <= 7 And | | | | |
| | ASPARTATE | percentile | <=65th percentile | | |
| | AMINOTRANSFERASE = Y) | | | | |
| 87 | (TISSUE = LIVER And | GLUCOSE <= | GLUCOSE <= 65th | all else | 4.15 |
| | TimePoint >= 5 | 5th percentile | percentile And | | |
| | but <= 7 And | | GLUCOSE >= | | |
| | GLUCOSE = Y) | | 35th percentile | | |
| 88 | (TISSUE = LIVER And | Day5_Logratio_ALP + | Logratio_ALP + | all else | 4.31 |
| | TimePoint >= 0.25 | Logratio_ALT >= | Logratio_ALT <= | | |
| | but <= 7 And | 90th percentile | 60th percentile | | |
| | Day5_Logratio_ALP + | | | | |
| | Logratio_ALT = Y) | | | | |
| 89 | (Tissue = LIVER) | >=0.0000000000001 | −3 | All else | 4.63 |
| 90 | (Tissue = LIVER And | (STRUCTURE_{—} | All else | (Zero_Class = | 6.45 |
| | HighOrLowDose = HI) | ACTIVITY = | | ***Blank***) Or | |
| | Not (STRUCTURE_{—} | Sterol 14-demethylase | | (Zero_Class = Y) | |
| | ACTIVITY = | inhibitor, miconazole | | | |
| | ***Blank***) | like | | | |
| 91 | (Tissue = LIVER) | >=0.0000000000001 | −3 | All else | 4.37 |
| 92 | (Tissue = LIVER) | >−1 Not ***Blank*** | −3 | All else | 4.63 |
| 93 | (Tissue = LIVER) | >−1 Not ***Blank*** | −3 | All else | 4.10 |
| 94 | (Tissue = LIVER And | (ACTIVITY_CLASS_{—} | All else | (Zero_Class = | 5.18 |
| | HighOrLowDose = HI) | UNION = | | ***Blank***) Or | |
| | Not (ACTIVITY_CLASS_{—} | Sterol 14-demethylase | | (Zero_Class = Y) | |
| | UNION = | inhibitor_Sterol | | | |
| | ***Blank***) | 14-demethylase | | | |
| | | inhibitor, ketoconazole | | | |
| | | like_Sterol | | | |
| | | 14-demethylase inhibitor, | | | |
| | | miconazole like | | | |
| 95 | (TISSUE = LIVER And | LIVER-FATTY CHANGE | LIVER-FATTY CHANGE | all else | 5.32 |
| | TimePoint >= 5 | SEVERITY SCORE > 2 in | SEVERITY SCORE = | | |
| | but <= 7 And | at least 3 animal(s) | 0 in all animals | | |
| | LIVER-FATTY | | | | |
| | CHANGE = Y) | | | | |
| 96 | (Tissue = LIVER) | hi dose PXR (clotrimazole, | other liver | BLIND, AVENTIS LOW | 8.52 |
| | | miconazole, mifepristone, | | DOSE and ALL OTHER | |
| | | dexamethansone) KYLE | | timeponts for 1 s | |
| 97 | (Tissue = LIVER) | (PXR_Class_1_{—} | (PXR_negative_{—} | All else | 4.61 |
| | | NO_DEX = YES) | specific = | | |
| | | | YES) Or (mifepristone | | |
| | | | included = | | |
| | | | EITHER + OR −) | | |
| 98 | (Tissue = LIVER) | (PXR_Class_1_all = | (PXR_negative_class_{—} | All else | 5.05 |
| | | YES) | large = YES) | | |
| 99 | (Tissue = LIVER) | (PXR_Class_1_DOSE = | (PXR_negative_ligand_{—} | All else | 4.71 |
| | | HI) | CYP 3A_inhibitors_{—} | | |
| | | | literature = YES) | | |
| 100 | (Tissue = LIVER) | (PXR_Class_1_DOSE = | (PXR_negative_specific = | All else | 11.15 |
| | | HI) | YES) | | |
| 101 | (Tissue = LIVER And | 98th percentile; | 0-75th percentile; | other | 5.71 |
| | TimePoint >= 5 And | liver; day 5/7 | liver; day 5/7 | | |
| | Organ_Weight_Info = | | | | |
| | Y) | | | | |
| 102 | (Tissue = LIVER) | (STRUCTURE_{—} | All else | (Zero_Class = | 5.00 |
| | Not (STRUCTURE_{—} | ACTIVITY = | | ***Blank***) Or | |
| | ACTIVITY = | DNA-Polymerase | | (Zero_Class = Y) | |
| | ***Blank***) | Inhibitor, thiopurine | | | |
| | | base | | | |
| 103 | (TISSUE = LIVER And | LEUKOCYTE | LEUKOCYTE COUNT >= | all else | 4.42 |
| | TimePoint >= 3 | COUNT <= 5th | 35th percentile | | |
| | but <= 7 And | percentile | | | |
| | LEUKOCYTE | | | | |
| | COUNT = Y) | | | | |
| 104 | (Tissue = LIVER And | 98th % | 25-75th % | rest | 4.89 |
| | TimePoint >= 5 And | | | | |
| | ClinicalChemInfo = Y) | | | | |
| 105 | (TISSUE = LIVER And | Day5_LIVER-NECROSIS | Day5_LIVER-NECROSIS | all else | 5.17 |
| | TimePoint >= 0.25 | SUM OF SEVERITY | SUM OF SEVERITY | | |
| | but <= 1 And | SCORE > 2 | SCORE = 0 | | |
| | Day5_LIVER- | | | | |
| | NECROSIS = Y) | | | | |
| 106 | (TISSUE = LIVER And | Day5_LIVER- | Day5_LIVER- | all else | 4.68 |
| | TimePoint >= 0.25 | PERITONITIS | PERITONITIS SUM | | |
| | but <= 1 And | SUM OF SEVERITY | OF SEVERITY | | |
| | Day5_LIVER- | SCORE > 0 | SCORE = 0 | | |
| | PERITONITIS = Y) | | | | |
| 107 | (Tissue = LIVER And | (ACTIVITY_CLASS_{—} | All else | (Zero_Class = | 4.88 |
| | HighOrLowDose = HI) | UNION = | | ***Blank***) Or | |
| | Not (ACTIVITY_CLASS_{—} | NSAID, COX-1_NSAID, COX-1, | | (Zero_Class = Y) | |
| | UNION = | 6-Methoxy-naphthalenyl- | | | |
| | ***Blank***) | acetic acid_NSAID, | | | |
| | | COX-1, arylacylprofen_{—} | | | |
| | | NSAID, COX-1, ibuprofen | | | |
| | | like_NSAID, COX-1, | | | |
| | | indomethacin like | | | |
| 108 | (Tissue = LIVER And | 95th % | 0-75th % | rest | 4.01 |
| | TimePoint <= 1 And | | | | |
| | ClinicalChemInfo = Y) | | | | |
| 109 | (Tissue = LIVER And | subcutaneous administra- | ALL ELSE | BLIND, AVENTIS | 5.20 |
| | TimePoint >= 3 | tion and LIVER repid, | | | |
| | but <= 5) | d3 and d5 | | | |
| 110 | (TISSUE = LIVER And | HEMOGLOBIN <= | HEMOGLOBIN >= | all else | 4.23 |
| | TimePoint = 3 And | 5th percentile | 35th percentile | | |
| | HEMOGLOBIN = Y) | | | | |
| 111 | (Tissue = LIVER And | 98th % | 25-75th % | rest | 4.40 |
| | TimePoint >= 5 And | | | | |
| | ClinicalChemInfo = Y) | | | | |
| 112 | (TISSUE = LIVER And | ABSOLUTE SEGMENTED | ABSOLUTE SEGMENTED | all else | 4.25 |
| | TimePoint >= 5 | NEUTROPHIL >= | NEUTROPHIL <= | | |
| | but <= 7 And | 95th percentile | 65th percentile And | | |
| | ABSOLUTE SEGMENTED | | ABSOLUTE SEGMENTED | | |
| | NEUTROPHIL = Y) | | NEUTROPHIL >= | | |
| | | | 35th percentile | | |
| 113 | (TISSUE = LIVER And | LYMPHOCYTE <= | LYMPHOCYTE >= | all else | 4.64 |
| | TimePoint >= 3 | 5th percentile | 35th percentile | | |
| | but <= 7 And | | | | |
| | LYMPHOCYTE = Y) | | | | |
| 114 | (TISSUE = LIVER And | Day5_Logratio_TBI + | Logratio_TBI + | all else | 4.50 |
| | TimePoint = 3 And | Logratio_ALP + | Logratio_ALP + | | |
| | Day5_Logratio_TBI + | Logratio_ALT <= | Logratio_ALT >= | | |
| | Logratio_ALP + Logratio_{—} | 5th percentile | 35th percentile | | |
| | ALT = Y) | | | | |
| 115 | (TISSUE = LIVER And | ALBUMIN <= | ALBUMIN >= | all else | 6.68 |
| | TimePoint = 3 And | 5th percentile | 35th percentile | | |
| | ALBUMIN = Y) | | | | |
| 116 | (Tissue = LIVER And | 95th percentile; | 0-75th percentile; | other | 4.33 |
| | TimePoint >= 5 And | liver; day 5/7 | liver; day 5/7 | | |
| | ClinicalChemInfo = Y) | | | | |

### Example 3

### Generation of Reduced Subsets of Genes Based on Impact Factor

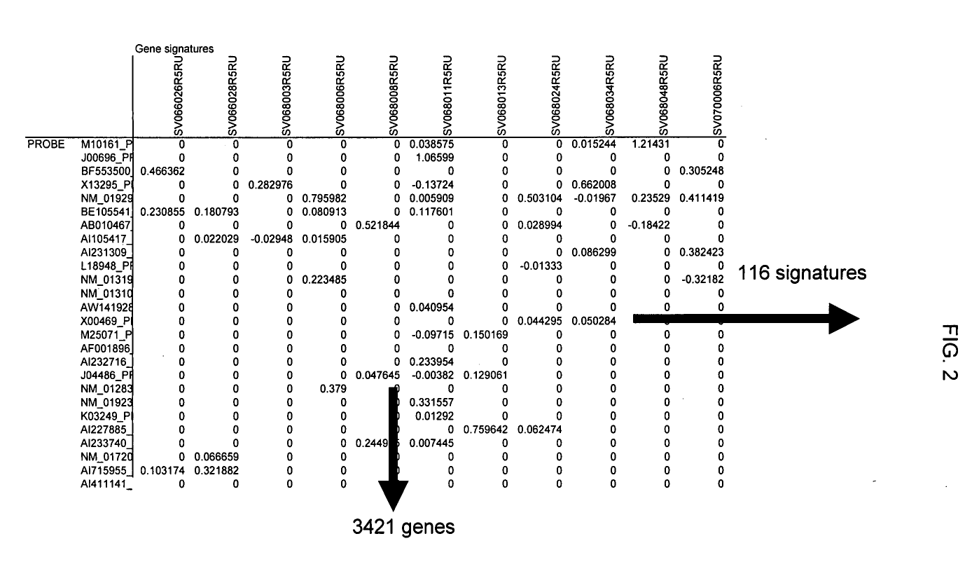

Each of the 116 non-redundant gene signatures listed in Table 3 above was broken down into its constituent variables (i.e. a total of 3421 different genes) and assembled in a single table of genes versus signatures. The weighting factors associated with each gene in each signature were inserted in the cells of the table. The “impact factor” (i.e., the product of the expression logratio and weighting factor) was calculated for each of the 3421 genes in each of the 116 signatures, and a table of identical dimensions was constructed. FIG. 2 shows a section of the complete 3421×116 impact factor table. The impact factor table is sparse (i.e. includes a large number of zero value entries) because the average number of genes in each signature with a non-zero weighting factor is on the order of 50 or less. A cursory examination of the abbreviated impact table of FIG. 2 reveals that a few of the genes appear multiple times across the spectrum of different signatures, whereas the majority of genes appear in just one or very few signatures. This observation suggests that subsets of fewer than the 3421 genes exist that may be sufficient to answer all of the 116 non-redundant (and 439 redundant) classification questions posed to the 8565 genes represented in the full rat liver tissue chemogenomic dataset.

A total impact factor was calculated for each of the 3421 genes across all 116 signatures. All of the 3421 genes were then ranked based on its total impact factor. The list ranking all 3421 is shown in Table 4 (included as the ASCII formatted file named “Table_{—}4.txt” included on the accompanying CD, which is hereby incorporated by reference herein). Using this ranking table, reduced subsets of genes consisting of the top ranking 100, 200, 400, 800, and 1600 genes from the set of 3421 were selected.

Based on publicly available annotation information regarding the 3421 genes in the reduced subset depicted in Table 4 (included as the ASCII formatted file named “Table_{—}4.txt” included on the accompanying CD), an additional subset corresponding to genes for secreted proteins in the reduced subset also were identified and listed in Table 5.

TABLE 5
Subset of Secreted Proteins from 3421 Gene LIVER Dataset

| Accession# | Rank |
|---|---|
| X13295 | 3 |
| AI231309 | 5 |
| NM_019237 | 17 |
| AW141928 | 18 |
| D86345 | 20 |
| AF117820 | 21 |
| NM_012552 | 24 |
| NM_012834 | 25 |
| U20194 | 31 |
| AI176002 | 32 |
| BF405996 | 36 |
| J04486 | 38 |
| AA894092 | 46 |
| AI406469 | 47 |
| BE108381 | 72 |
| NM_017208 | 85 |
| AF187814 | 88 |
| AA819103 | 94 |
| M55601 | 103 |
| AI170387 | 143 |
| U48245 | 144 |
| D14839 | 155 |
| U06436 | 171 |
| AB036792 | 184 |
| AJ001044 | 188 |
| L20869 | 195 |
| Y00480 | 200 |
| U38379 | 220 |
| AW253902 | 222 |
| AA874924 | 245 |
| AA799400 | 254 |
| BF409208 | 259 |
| AF030378 | 262 |
| NM_013042 | 264 |
| Z49761 | 271 |
| D88666 | 281 |
| BF282574 | 286 |
| L06238 | 319 |
| U67914 | 320 |
| NM_012588 | 397 |
| BE109691 | 427 |
| AA892897 | 450 |
| U69278 | 496 |
| BF420018 | 508 |
| BF563403 | 545 |
| AI172159 | 548 |
| AF010466 | 559 |
| NM_016998 | 582 |
| Y00697 | 602 |
| AA866419 | 622 |
| NM_012835 | 633 |
| AW434520 | 654 |
| BF558479 | 656 |
| U02983 | 657 |
| AF276940 | 671 |
| AF058786 | 689 |
| AF312687 | 692 |
| BF282313 | 716 |
| AF171936 | 719 |
| BF562675 | 725 |
| AW915518 | 732 |
| AF007818 | 734 |
| BE111752 | 759 |
| AF245040 | 761 |
| M63574 | 771 |
| D00036 | 782 |
| NM_013174 | 784 |
| M31155 | 800 |
| AI144646 | 830 |
| BF281544 | 857 |
| BE101448 | 859 |
| M31176 | 909 |
| X13309 | 934 |
| AI009783 | 938 |
| AF109643 | 955 |
| AA943794 | 957 |
| L36459 | 970 |
| NM_012777 | 997 |
| M22899 | 999 |
| NM_019205 | 1019 |
| AI146056 | 1036 |
| AI230918 | 1045 |
| U32679 | 1055 |
| AA800483 | 1057 |
| M34643 | 1061 |
| AI599031 | 1062 |
| BF557871 | 1064 |
| AI411222 | 1072 |
| U04319 | 1075 |
| NM_019258 | 1080 |
| NM_013413 | 1081 |
| U03491 | 1093 |
| AI406660 | 1120 |
| AI012235 | 1126 |
| BF281577 | 1161 |
| AF149118 | 1163 |
| BF282961 | 1210 |
| NM_017113 | 1255 |
| AW251324 | 1278 |
| AI176773 | 1287 |
| AF177031 | 1297 |
| AF110024 | 1300 |
| AW524733 | 1309 |
| M98820 | 1326 |
| NM_019199 | 1327 |
| D38494 | 1330 |
| NM_012916 | 1332 |
| M15797 | 1338 |
| D12678 | 1381 |
| U00620 | 1395 |
| AI236084 | 1424 |
| NM_012679 | 1449 |
| AI072892 | 1455 |
| BF522885 | 1500 |
| U04317 | 1516 |
| X89963 | 1531 |
| AF062402 | 1533 |
| S57864 | 1574 |
| BF396114 | 1581 |
| NM_019192 | 1597 |
| U56859 | 1613 |
| AI070123 | 1622 |
| AI104235 | 1624 |
| Z78279 | 1631 |
| AF121670 | 1643 |
| AF193014 | 1654 |
| AF153012 | 1694 |
| BE106542 | 1698 |
| BE109018 | 1704 |
| AI716642 | 1715 |
| AA891826 | 1721 |
| NM_012707 | 1776 |
| BE096501 | 1797 |
| NM_019153 | 1840 |
| AW918222 | 1848 |
| NM_017139 | 1862 |
| NM_012881 | 1867 |
| X13722 | 1884 |
| AF163569 | 1903 |
| U48246 | 1952 |
| AW142280 | 1975 |
| U07615 | 1997 |
| AW142880 | 2015 |
| J04811 | 2016 |
| BF415013 | 2026 |
| NM_012549 | 2037 |
| X82152 | 2074 |
| AF053312 | 2096 |
| U62667 | 2123 |
| AI227829 | 2130 |
| M64711 | 2133 |
| NM_012493 | 2137 |
| C06844 | 2141 |
| AB022883 | 2150 |
| NM_017310 | 2152 |
| AI412180 | 2178 |
| AA850725 | 2182 |
| AW915104 | 2199 |
| AI070137 | 2208 |
| AJ011811 | 2210 |
| AF259981 | 2223 |
| AI236616 | 2275 |
| J03624 | 2280 |
| AI407409 | 2304 |
| AI013906 | 2306 |
| U67884 | 2318 |
| NM_012715 | 2328 |
| BF420163 | 2342 |
| D83231 | 2372 |
| AA892824 | 2375 |
| D10763 | 2378 |
| AF177430 | 2384 |
| BE349699 | 2398 |
| X59290 | 2421 |
| NM_019150 | 2422 |
| NM_017094 | 2436 |
| AF221622 | 2465 |
| BF416115 | 2491 |
| NM_012614 | 2528 |
| AI102026 | 2553 |
| AJ299016 | 2563 |
| AW434178 | 2578 |
| NM_012974 | 2600 |
| AA891690 | 2610 |
| AF093567 | 2624 |
| AI411527 | 2671 |
| AF068861 | 2684 |
| BE108973 | 2734 |
| AF159103 | 2746 |
| AI412418 | 2754 |
| NM_012738 | 2759 |
| NM_017066 | 2760 |
| X59859 | 2811 |
| X92495 | 2823 |
| AA892798 | 2859 |
| AI179372 | 2877 |
| U94856 | 2889 |
| NM_012553 | 2939 |
| U66566 | 2956 |
| L04796 | 2969 |
| NM_013139 | 2970 |
| U59672 | 2977 |
| U22520 | 2996 |
| M58716 | 3011 |
| NM _017110 | 3070 |
| BF416285 | 3075 |
| BF567631 | 3102 |
| AF323085 | 3109 |
| M88469 | 3124 |
| AF093536 | 3143 |
| Y11490 | 3158 |
| AI232348 | 3187 |
| NM_017292 | 3212 |
| D00403 | 3219 |
| NM_019322 | 3222 |
| X66539 | 3245 |
| AF014827 | 3253 |
| AF188608 | 3312 |
| AF047707 | 3333 |
| AA944398 | 3338 |
| NM_017259 | 3340 |
| AW917069 | 3415 |

### Example 4

### Validating Reduced Subset Performance

This example illustrates how reduced subsets of 3421, 800, or even just 100, genes made according to Examples 1-3, may be used to generate new versions of the 116 signatures capable of performing liver tissue chemogenomic classification tasks with comparable, or better performance to the original set of 8565 genes.

A. Validating that Subsets of Genes are “Sufficient”

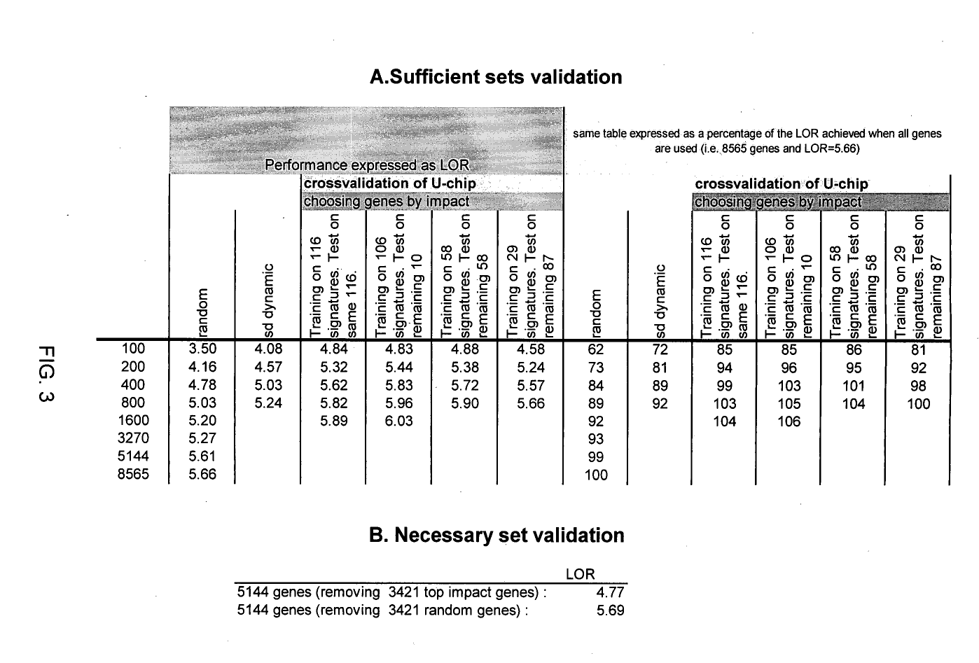

The 116 non-redundant signatures for the rat liver dataset described above were regenerated and three-fold cross-validated using only reduced subsets of gene of varying size as the input variables (FIG. 3). Signature performance was defined as average test LOR for all 116 three-fold cross-validated signatures (see values in left portion of table depicted in FIG. 3A). Performance also was expressed as a percentage of the maximum LOR achieved when all 8565 genes present on the chip were used to generate the 116 signatures (see values in right portion of the table depicted in FIG. 3A).

For comparison purposes, results also were obtained with gene subsets of similar sizes chosen either randomly, or based on the standard deviation of their log-ratio across all treatments under considerations of a given signature. Gene selection based on standard deviation results in gene subsets including those genes showing the highest variability across the dataset. As shown in FIG. 3, the standard deviation (sd) based gene choice always performs better than random gene choice.

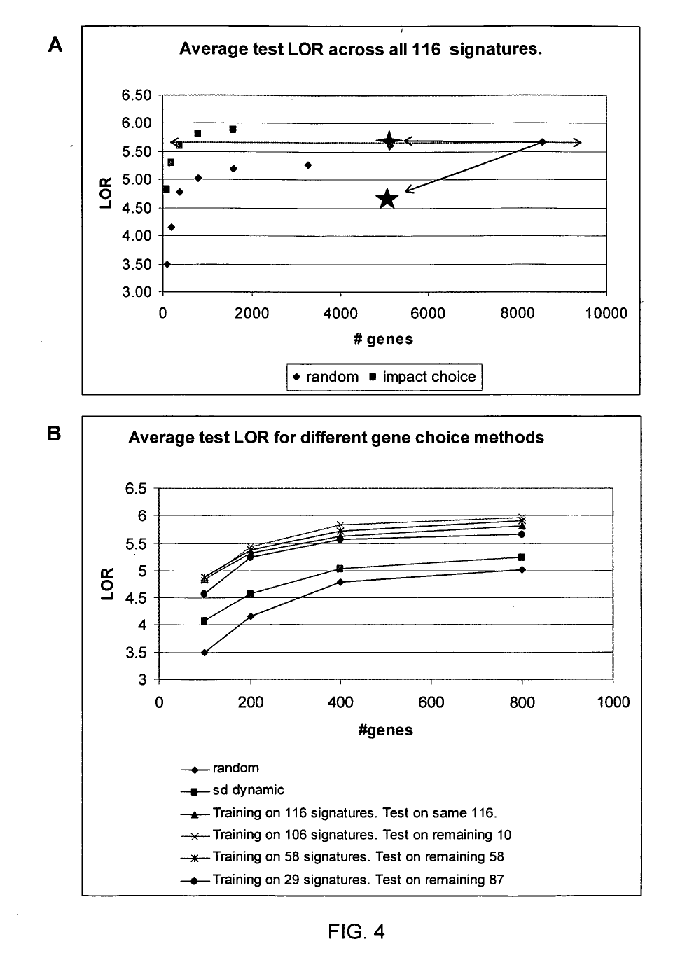

As illustrated by the results in FIG. 3A, even just 100 of the genes with the highest impact factors are sufficient to achieve an average logodds ratio (LOR) of 4.84. All the data in FIGS. 3, 4A and 4B are averages over 116 signatures of the three-fold cross-validated test logodds ratio. This LOR value corresponds to a performance level that is 85% of the maximum achieved when all 8565 genes are used to generate the 116 signatures (LOR=5.66). Thus, a specific reduced subset using only about 1% of the total number of genes in the full dataset of 8565, can achieve 85% of the full sets performance for chemogenomic classification tasks.

Significantly, a slightly larger subset of just 800 high impact genes is sufficient to achieve an average performance of LOR=5.82, or 103% of the performance achieved when all 8565 genes are used. Thus, a specific reduced subset including <10% of the number of genes as in the full dataset of 8565, is “sufficient” to achieve maximum classification performance. It should be noted that this cross-validation analysis used the same 116 questions that were used to derive the first set of linear classifiers from the complete dataset.

The 800 gene subset described above is not unique in its ability to classify the complete dataset. When weighting factors alone, rather than impact factor, were used to select genes the resulting 800 gene subset does not completely overlap with the impact-factor based 800 gene subset. Regardless, the weight-based 800 gene subset was found to produce similar results in terms of performance.

B. Generating Non-Overlapping “Sufficient” Sets of Genes

As shown above, a sufficient set of 100 genes with LOR=4.84 may be generated. An interesting question is whether a completely different (i.e. non-overlapping) sufficient set of genes with equal performance may also be generated from the full dataset. Given that the first set of 100 genes is the best set derived according to our method, the other sets will probably need to be larger. A simple method for deriving a non-overlapping set is to test the performance of the next 100-200 genes in the impact ranked list of 3421 genes. Table 6 compares the performance of the first 100 genes, LOR=4.84, to that of the next 100 genes, LOR=4.42.

TABLE 6
Comparison of Non-Overlapping Gene Sets
genes are all chosen from the list of 3421 genes ranked by impact

| | | ave test LOR |
|---|---|---|
| number of genes | rank | (116 signatures) |
| 100 | 1-100 | 4.84 |
| 100 | 100-200 | 4.42 |
| 200 | 100-300 | 4.95 |
| 300 | 100-400 | 5.24 |

As shown in Table 6, the set of the next 100 ranked genes is completely non-overlapping with the first and has a lower performance. However increasing the number of genes to 200 or 300 creates gene sets with a performance higher than the original set. Thus, at least two sufficient gene sets have been generated by the method of the invention (i.e. the last two lines in Table 6) that are non-overlapping with the first set. Each is sufficient to perform with a LOR>4.84.

This example illustrates that alternate non-overlapping “universal” gene sets exist for any given performance threshold. This, leads to the question answered below: “What is the set of all genes capable of LOR>4.84?”

C. Validating that the Subset of 3421 Genes Constitutes a “Necessary” Set

A “necessary” gene list was defined empirically as the list of genes, N, chosen from the list of all genes present in the dataset, A, such that the performance of the remaining genes, R (where R=A-N), fails to rise above some threshold. In the present example, the level of performance was defined as that achieved by the smallest “sufficient” gene set identified according to the methods described above. Specifically, the 100 gene subset chosen using the impact factor based method that achieves an LOR of 4.84 (see, FIG. 3A).

Confirmation that the subset of 3421 genes was “necessary” was carried out as follows. The set of 3421 genes was removed from the complete set of 8565 genes (i.e. the 8565 genes listed in Table 7, included as ASCII formatted file named, “Table_{—}7.txt” on accompanying CD, which is hereby incorporated by reference herein). All 116 of the originally derived non-redundant signatures were recomputed using the “depleted” subset consisting of the remaining 5144 genes (i.e. 8565-3421=5144) as the input dataset. These signatures were evaluated using the same cross validation procedure. In addition, as a control, a random set of 3421 genes was also removed producing a set of random 5144 genes. As shown by the values in the table depicted in FIG. 3B, removing the specific 3421 genes in the “necessary” subset of top impact genes results in a subset of 5144 genes that performs worse (LOR=4.77) than even the small “sufficient” subset of 100 genes selected based on impact factor (LOR=4.84).

Furthermore, removal of 3421 random genes was found to have no substantial effect on the performance of the remaining 5144 gene subset (LOR=5.69) relative to the full set of 8565 (LOR=5.66). The effect of the removal of the specific subset of 3421 high impact genes is further illustrated by the decrease in performance of the remaining 5144 gene subset represented by the two stars in FIG. 4A.

Because the specific set 5144 genes remaining after removal of the 3421 high impact genes cannot produce a signature with a minimal threshold performance LOR>4.84, it was concluded that the 3421 genes constitute a “necessary” subset.

### Example 5

### Validating Performance of Reduced Gene Sets for Generating Novel Signatures

This example illustrates a simulation demonstrating the ability of reduced gene sets to answer novel queries (i.e., generate signatures capable of answering chemogenomic classification questions not posed to the original dataset).

Reduced subsets of 100, 200, 400, 800, and 1600 genes from the full set of 8565 genes were identified based on the methods described in Examples 1-4, but using only a random subset of 106 out of the complete set of 116 non-redundant signatures. Reduced gene subset selection was based on impact factor ranking as described in Example 3. The 100, 200, 400, 800, and 1600 gene subsets were then used as input to generate the remaining 10 signatures that had not been used to generate the subsets.

The performance of each reduced subset was defined as the average of the test LOR (three-fold cross validated) for the remaining 10 signatures so generated. This procedure was repeated systematically for a total of ten different 106/10 splits of the 116 signatures. This same “split-sample” cross validation procedure then was repeated for different split ratios of the 116 signatures (e.g. 58/58 and 29/87).

As shown by the data presented in FIG. 3, all four reduced subsets perform comparably to, or even better than, the complete set of 8565 genes for the simulated task of identifying signatures for novel classification questions (and much better than randomly selected subsets, or subsets selected based on high variability of the selected genes across all signatures i.e., “sd dynamic”). As shown more visually from the graph in FIG. 4B, all four curves plotting the performance of the high impact reduced subsets, with the possible exception of the one corresponding to the 29/87 split ratio, are indistinguishable. This result supports the conclusion that the reduced gene subsets made by the method described of the present invention have “universal” value; that is, they perform equally well on classification tasks that were, or were not, involved in deriving the genes in each subset.

Furthermore, examination of the four high impact gene subset cross validation curves (shown in FIG. 4B) reveals that the genes present in a random set of 106, or even 58, signatures contain enough information to answer previously unasked chemogenomic classification questions without a loss of performance relative to the full set of genes.

### Example 6

### Recalibration of Signatures for a New Diagnostic Device Using a Reduced Set of Chemogenomic Data

A large chemogenomic dataset comprising the expression levels of 8565 genes in response to 311 compounds may be mined to generate 439 signatures (for liver tissue). These signatures (i.e., linear classifiers which comprise genes and weights) are useful for classifying a wide range known or unknown compound treatments. However, the full set of 8565 genes is not necessary to carry out most chemogenomic classification tasks. As shown in Examples 1-5, a non-redundant subset of 116 signatures may be mined to derive a subset of 3421 (or even fewer) information rich genes that effectively provide the bulk of the genomic responsiveness necessary to carry out all of the classification tasks. In other words, a subset of only 3421 or fewer of the original 8565 genes may be used to carry out all of the chemogenomic classification tasks with as good a level of performance. Thus, as described in Example 7, greatly simplified chemogenomic analysis devices (e.g., DNA microarrays) may be prepared using reagent sets directed to the reduced subset of genes. These simplified devices should provide comparable performance at higher throughput and lower cost. However, if the simplified device based on the reduced set of genes is not based on the same device platform as used to generate the original multivariate chemogenomic dataset, it may be necessary to optimize or recalibrate the signatures for the new platform.

Recalibration to a new platform requires running new chemogenomic assays on that platform and re-generating the signatures. However, as is shown in this Example, the data regeneration process may be greatly abbreviated and still result in a set of signatures capable of performing at a level as good as those derived based on a much larger dataset.

A large chemogenomic dataset was assembled that included measurement of expression levels in liver tissue for 8565 different genes on an Amersham CodeLink RU1 microarray platform in response to 1658 different compound treatments at varying dosages and time points. A set of 175 non-redundant signatures (i.e., classifiers) was generated and used to identify a necessary subset of 400 highly informative genes in liver tissue according to the methods described in Examples 1-5.

For purposes of choosing a method capable of identifying the most informative treatments the original chemogenomic dataset of 1658 compound treatments was split into a “training” set of 1279 treatments and “test” set of 320 treatments (59 treatments were not included in the training set because they were not labeled as either in a positive or negative class for any of the signatures). The split of treatments between the training and test set was made so as to insure that treatments from both the positive and negative classes for each signature were represented in both the training and test sets. In addition, all 175 signatures were generated based on sets of compound treatments wherein the minimum size for the positive class was six treatments.

In splitting the set of 1658 treatments into the training and test sets, the set of compound treatments for each signature was considered successively. For each signature, two of the positive class treatments were chosen randomly and assigned to the test set. This random selection method resulted in 320 treatments in the test set. This number was less than twice the total number of signatures (i.e., 350) because some of the randomly selected treatments were in the positive class for more than one signature. The negative class for the test set was defined as the non-redundant union of the positive classes for all other signatures. Designing the training/test split in this manner ensured that it was always possible to evaluate a signature on the test set of compound treatments using the LOR.

The original set of 175 non-redundant signatures were re-generated using only the 1279 “training set” treatments or some percentage subset of these 1279 treatments selected according to one of the three methods as described below. The performance of these re-generated signatures was then determined by classifying the “test set” of 320 treatments.

Method 1

Method 1 is based on the observation that the negative class (i.e., set of “−1” labelled treatments) of many signatures is much larger than the positive class (i.e., +1 labelled treatments), and thus, many treatments in the negative class may be eliminated as redundant. Three different variants of Method 1 were used and all resulted in treatment sets of reduced size.

In the first version of method 1 (“method 1_{—}1”) all treatments that only appear in the negative class and never in the positive class for any of the 175 signatures were eliminated. This resulted in a set of only 818 treatments (i.e., 64% of the 1279). The 175 signatures were regenerated using only expression levels for the reduced subset of 400 highly informative genes in response to this subset of 64% of the original treatments. The performance of these regenerated signatures was then measured by classifying the 320 compound “test set” treatments. This performance was compared to that of the 175 signatures re-generated using the expression of the 400 gene subset but the full “training set” of 1279 compound treatments. It was found that the 175 signatures based on measurements using only the 64% of compound treatments (identified by label trimming according to Method 1_{—}1) actually performed with an average logodds ratio of 4.61, slightly higher than the 4.58 value measured for the signatures based on the full treatment set. This demonstrates that re-calibration of signatures for a different device platform may be carried out based on a greatly reduced set of new chemogenomic measurements.

Further reductions in the amount of new data collected may be achieved according to a further variant of Method 1. This second variation is based on the fact that there is a subset of treatments that appear only in signatures with a large positive class. By removing half (Method 1_{—}2) or all (Method 1_{—}3) of these large positive class treatments it is possible to further reduce the number of compound treatments and generate a set of 175 re-calibrated signatures (based on the 400 genes) that maintain a high level of performance relative the signatures generated using the full set of 1279 treatments. Method 1_{—}2 requires only 43% of the 1279 treatments but yields a set of 175 signatures that classify the “test set” with an average LOR of 4.38. Label trimming based on Method 1_{—}3 results in only 24% of the 1279 treatments, but the resulting 175 signatures perform with an average LOR of 4.16. These results regarding performance indicate that one may re-calibrate a set of signatures for chemogenomic analysis for use on a new device platform (e.g., go from a microarray to a RT-PCR device) and carry out only a fraction of the original measurements.

Two other methods for reducing the number of treatments necessary for signature recalibration have been tested. Method 2 is based on the assumption that those compound treatments closest to the boundary between the two classes are the most important to define the entire class. These “border lining” treatments are easily identified for a given signature by the fact that their Scalar Product (SP) is close to +1 or −1 for the positive and negative classes, respectively. Using this method, different portions of the training set corresponding to 39%, 31% and 29% of the 1279 treatments were selected and used to regenerate the 175 signatures. However, the performance of these signatures were significantly poorer (avg. LOR=3.52, 3.52 and 3.54, respectively) than that exhibited by Method 1. The poorer performance of this method probably indicates the weakness of the assumption that those treatments lining the inner borders of the classes are more significant. Indeed, it may be that these boundary treatments are often outliers or even possibly mislabeled.

Like Method 2, Method 3 is based on identifying those treatments most significant for defining the class boundary, however, Method 3 utilizes Support Vector Machines (SVM) methods and yields performance even higher than Method 1 for re-generating signatures. According to Method 3, a set of most informative compound treatments is derived based on their relative importance to defining the linear decision boundary between the class of positive and negative treatments for each of the 175 signatures. The linear decision boundary is determined using a linear kernel an Adjusted Kernel Support Vector Machine (A-K-SVM) algorithm. This method relies on one of the key characteristics of the use of SVMs to define classifiers: the resulting decision boundary is described entirely by only a subset of all of the treatments considered for a given signature. This subset that defines the boundary are called the support vectors, and with each of these support vector is associated a support value. The support values may be used to determine how important the corresponding treatment is to describe the decision boundary accurately.

According to Method 3, the subset of the most relevant treatments for the set of 175 signature was derived from a ranking the sum of the support values (rescaled within [0,1]; 0 if it is not a support vector) for each of the signatures where the treatment is considered, and dividing this sum by the total number of signatures for which the treatment is considered. After removing treatments that only appear in negative classes, the set of the N most relevant treatments was constructed by removing from the remaining treatments those with the lowest ranking. However, if removing a treatment reduces any of the positive classes (for all signatures) to less than 3 treatments, the treatment is not removed. The removal process stops when N treatments remain.

Method 3 was used to select two different treatment subsets of 53% and 38% of the full set of 1279 treatments. The specific subset of 53% of all treatments was able to re-generate the 175 signature with no loss in performance relative to full treatment set (avg. LOR=4.59). Moreover, the specific subset of treatments selected according to Method 3 that included only 38% of the 1279 exhibited only a slight degradation in performance (avg. LOR=4.51).

### Example 7

### Construction of a “Universal” Rat Liver Tissue DNA Array

The reduced subset of 800 “sufficient” genes selected according to Examples 1-4 described above is used as the starting point for building an 800 oligonucleotide probe DNA array. The probe sequences used to represent the 800 genes on the array are the same ones used on the CodeLink® RU1 DNA array described in Table 7 (which is disclosed in the ASCII formatted file named “Table_{—}7.txt” included on the accompanying CD, which is hereby incorporated by reference herein). The 800 probes are pre-synthesized in a standard oligonucleotide synthesizer and purified according to standard techniques. The pre-synthesized probes are then deposited onto treated glass slides according to standard methods for array spotting. Large numbers of slides, each containing the set of 800 probes, are prepared simultaneously using a robotic pen spotting device as described in U.S. Pat. No. 5,807,522. Alternatively, the 800 probes may be synthesized in situ on one or more glass slides from nucleoside precursors according to standard methods well known in the art such as ink-jet deposition or photoactivated synthesis.

The 800 probe DNA arrays are then each hybridized with a fluorescently labeled sample derived from the mRNA of a compound treated rat's liver tissue according to the methods described in Example 1 above. The fluorescence intensity data from each array hybridization is used to calculate gene expression log ratios for each of the 800 genes. The log ratios are then used in conjunction with the chemogenomic dataset constructed as in Example to answer any of the 439 classification questions that may be relevant for the specific compound.

All publications and patent applications cited in this specification are herein incorporated by reference as if each individual publication or patent application were specifically and individually indicated to be incorporated by reference.

Although the foregoing invention has been described in some detail by way of illustration and example for clarity and understanding, it will be readily apparent to one of ordinary skill in the art in light of the teachings of this invention that certain changes and modifications may be made thereto without departing from the spirit and scope of the appended claims.

What is claimed is:

1. A method for preparing a high-throughput chemogenomic assay reagent set comprising:
a. deriving a set of non-redundant classifiers, each comprising a plurality of genes, from a chemogenomic dataset, wherein the chemogenomic dataset comprises expression levels for a plurality of gene measured in response to a plurality of compound treatments;
b. ranking each gene in the set of non-redundant classifiers based on its contribution across all of the non-redundant classifiers;
c. selecting the subset of genes ranking in about the 50^{th} percentile or higher; and
d. preparing a plurality of isolated polynucleotides or polypeptides, wherein each polynucleotide or polypeptide is capable of detecting at least one gene of the selected subset.

2. The method of claim 1, wherein the chemogenomic dataset comprises expression levels for at least 5000 genes.

3. The method of claim 1, wherein the chemogenomic dataset comprises at least about 100 different compound treatments.

4. The method of claim 1, wherein the set of non-redundant classifiers comprises at least about 50 classifiers.

5. The method of claim 1, wherein the selected subset of genes ranks in about the 90^{th} percentile or higher.

6. The method of claim 1, wherein the selected subset of genes comprises about 800 or fewer genes.

7. The method of claim 1, wherein the selected subset of genes comprises about 100 or fewer genes.

8. The method of claim 1, wherein the method of ranking the genes across all classifiers is selected from the group consisting of: determining the sum of weights; determining the sum of absolute value of weights; and determining the sum of impact factors.

9. The method of claim 1, wherein the redundancy of the classifiers is determined using a fingerprint of resulting classifiers against a set of reference treatments.

10. The method of claim 9, wherein the fingerprint is assessed using a hierarchical clustering method selected from the group consisting of: UPGMA and WPGMA.

11. A reagent set made according to claim 1.

12. The reagent set of claim 11, wherein the number of reagents in the subset is less than about 10% of the number of genes in the full chemogenomic dataset.

13. The reagent set of claim 11, wherein the number of reagents in the subset is less than about 5% of the number of genes in the full chemogenomic dataset.

14. The subset of claim 11, wherein the number of genes is 800 or fewer.

15. The subset of claim 11, wherein the number of genes is 400 or fewer.

16. An array comprising a reagent set made according to claim 1.

17. The array of claim 16, wherein the reagent set consists of polynucleotides capable of detecting the genes listed in Table 4.

18. The array of claim 16, wherein the reagent set consists of polynucleotides capable of detecting the top ranking 800 genes listed in Table 4.

19. The array of claim 16, wherein the reagent set consists of polypeptides each capable of detecting a secreted protein encoded by the genes listed in Table 5.

20. A reagent set for chemogenomic analysis of a compound treated sample, wherein the set comprises a plurality of polynucleotides or polypeptides, wherein each polynucleotide or polypeptide is capable of detecting at least one member of a subset of less than about 10 percent of the genes in a full chemogenomic dataset, and wherein the subset of genes is capable of generating a set of signatures that exhibit at least about 85 percent of the average performance of the same set of signatures generated from the full chemogenomic dataset.

21. The reagent set of claim 20, wherein the reagent set comprises a plurality of polynucleotides.

22. The reagent set of claim 21, wherein the plurality of polynucleotides are immobilized on one or more substrates.

23. The reagent set of claim 20, wherein the full chemogenomic dataset comprises expression levels for at least about 5000 genes.

24. The reagent set of claim 20, wherein the full chemogenomic dataset comprises at least about 100 different compound treatments.

25. The reagent set of claim 20, wherein the subset comprises less than about 5% of the genes in the full chemogenomic dataset.

26. The reagent set of claim 20, wherein the set of signatures comprises at least about 50 signatures.

27. The reagent set of claim 20, wherein the signatures are linear classifiers generated using support vector machines.

28. The reagent set of claim 20, wherein the subset is capable of generating a set of signatures that exhibit at least about 95 percent of the average performance of the same set of signatures generated from the full chemogenomic dataset.
